(19) United States
(12) Patent Application Publication
(10) Pub. No.: US 2009/0143276 A1
(43) Pub. Date: Jun. 4, 2009

(54) Trimeric IL-1Ra

(75) Inventors: Thor Las Holtet, Ronde (DK); Mikkel Holmen Andersen, Sporup (DK); John Nieland, Arhus C (DK)

Correspondence Address: MCDONNELL BOEHNEN HULBERT & BERGHOFF LLP, 300 S. WACKER DRIVE, 32ND FLOOR, CHICAGO, IL, 60606, US

(73) Assignee: Anaphore, Inc.

(21) Appl. No.: 12/247,897

(22) Filed: Oct. 8, 2008

Related U.S. Application Data
(60) Provisional application No. 60/978,254, filed on Oct. 8, 2007.

(51) Int. Cl. A61K 38/02; C07K 19/00; C12N 15/11; A61P 29/00; C12N 1/19; C12N 1/21; C12N 15/63; C12N 5/10

(52) U.S. Cl. 514/2; 530/300; 530/350; 530/410; 536/23.4; 435/320.1; 435/325; 435/252.3; 435/254.2; 435/235.1

(57) ABSTRACT

Interleuekin-1 receptor antagonists (IL-1Ra) including fusion proteins having a trimerizing domain and an IL-1Ra polypeptide sequence. The fusion proteins are part of trimeric complexes that are used in pharmaceutical compositions for treating diseases mediated by IL-1. Effective treatment of inflammatory diseases, such as rheumatoid arthritis and diabetes, are described.

### CROSS-REFERENCE TO RELATED APPLICATION

This application claims the benefit of U.S. Provisional Patent Application Ser. No. 60/978,254, filed Oct. 8, 2008, which is incorporated by reference herein in its entirety.

### FIELD OF THE INVENTION

The invention relates to treatment of diseases that are mediated by interleukin 1. More particularly, the invention relates to interleukin 1 receptor antagonists (IL-1Ra) that are useful for treating such diseases.

### BACKGROUND OF THE INVENTION

The IL-1 family is an important part of the innate immune system, which is a regulator of the adaptive immune system. The balance between IL-1 and IL-1Ra in local tissues influences the possible development of inflammatory diseases and resulting structural damage. In the presence of an excess amount of IL-1, inflammatory and autoimmune diseases may develop in the joints, lungs, gastrointestinal tract, central nervous system (CNS), or blood vessels. Treatment of human disease with IL-1Ra has been carried out through injection of recombinant IL-1Ra or through gene therapy approaches. Treatment with recombinant IL-1Ra has been approved for rheumatoid arthritis (RA) and phase 2 studies are ongoing for osteoarthritis (OA).

An important pro-inflammatory role for IL-1 in many human diseases has been described over the past 10 years. The balance between IL-1 and IL-1Ra has been extensively studied in a variety of animal disease models including rheumatoid arthritis (RA), osteoarthritis (OA), inflammatory bowel disease (IBD), granulomatous and fibrotic lung disorders, kidney diseases, diseases of the liver and pancreas, graft-versus-host disease (GVHD), leukemia, cancer, osteoporosis, diabetes, central nervous system diseases, infectious diseases, and arterial diseases. In each of these diseases, local overproduction of IL-1 and/or underproduction of IL-1Ra pre-disposes subjects to disease development. The therapeutic administration of IL-1Ra has been shown to be efficacious in preventing tissue damage (See W. P. Arend, Cytokine & Growth Factor Reviews, 13 (2002) pp. 323-240).

The IL-1 family consists of two agonists, IL-1α and IL-1β; the specific receptor antagonist IL-1Ra; and three different receptors, IL-1R type I (IL-1RI), IL-1R type II (IL-1RII) and IL-1 receptor accessory protein (IL-1R AcP). IL-1RI is an 80 kDa protein with a long cytoplasmic domain of 215 residues. The biologically inert IL-1RII is a 60 kDa protein with a short cytoplasmic domain of 29 residues. IL-1R AcP is recruited to the complex after binding of IL-1α or IL-1β to the single chain IL-1RI. Signal transduction pathways activated by the approximated cytoplasmic domains of IL-1RI and IL-1R AcP include the NF-κB, JNK/AP-1, and p38 MAP kinase pathways. IL-1RII functions as a decoy receptor, binding IL-1 both on the plasma membrane and as a soluble receptor in the fluid phase, thereby preventing IL-1 from interacting with the functional IL-1RI.

The third ligand in the IL-1 family, IL-1Ra, is a structural variant of IL-1 that binds to both IL-1R but fails to activate cells. IL-1Ra is a 17 kDa protein with 18% amino acid homology to IL-1α and 26% homology to IL-1β. The originally described isoform of IL-1Ra is secreted from monocytes, macrophages, neutrophils, and other cells and is now termed sIL-1Ra. Three additional intracellular isoforms of IL-1Ra have been described to date. An 18 kDa form of IL-1Ra, created by an alternative transcriptional splice mechanism from an upstream exon is called icIL-1Ra1 and is found inside keratinocytes and other epithelial cells, monocytes, tissue macrophages, fibroblasts, and endothelial cells. IL-1Ra cDNA cloned from human leukocytes contains an additional 63 bp sequence as an insert in the 5′ region of the cDNA. A 15 kDa isoform of IL-1Ra, termed icIL-1Ra3, is found in monocytes, macrophages, neutrophils, and hepatocytes, and may be created both by an alternative transcriptional splice as well as by alternative translational initiation.

Both soluble IL-1Ra and icIL-1Ra1 bind equally well to IL-1R, but icIL-1Ra3 exhibits weak receptor binding. IL-1Ra functions as a specific receptor antagonist by binding to IL-1RI but preventing IL-1R AcP from associating with the IL-1RI, which results failure of initiation of signal transduction pathways.

The decoy receptor IL-1RII binds IL-1 both on the plasma membrane and as a soluble receptor in the fluid phase, preventing IL-1 from interacting with the functional IL-1RI. Therefore, soluble IL-1RII and IL-1Ra can inhibit IL-1 in co-operation. Soluble IL-RI can bind to IL-1 as well as IL-1Ra, but due to the balance between IL-1 and IL-1Ra, soluble IL-1RI seems to act as a pro-inflammatory agent.

KINERET® is an E. coli produced IL-1Ra from Amgen, which has been shown to benefit patients with active rheumatoid arthritis. KINERET® has to be injected subcutaneously once per day. With subcutaneous administration, KINERET® has a half-life ranged from 4 to 6 hours; for i.v. administration the half life is approximately 2½ hours. IL-1Ra is cleared by renal clearance. KINERET® is a specific receptor antagonist of IL-1 that differs from naturally occurring IL-1 receptor antagonist by the presence of a methionine group. When given alone or in combination with methotrexate, KINERET® has been shown to benefit patients as assessed by improvement in clinical signs and symptoms, decreased radiographic progression and improvement in patient function, pain and fatigue. KINERET® has a favorable safety profile as demonstrated in clinical trials.

Several attempts have been made to improve the poor pharmacokinetics of IL-1Ra. Antibodies targeting just IL-1 beta have been developed. However, in contrast to IL-1ra, these only block IL-1 beta, but not IL-1α action.

Accordingly, the inventors have identified a need in the art for an improved delivery method for IL1-Ra, which provides for a longer half-life of the molecule and provides a favorable safety profile.

### BRIEF DESCRIPTION OF THE DRAWINGS

FIG. 1 shows alignment of the amino acid sequences of the trimerising structural element of the tetranectin protein family. Amino acid sequences (one letter code) corresponding to residue V17 to K52 comprising exon 2 and the first three residues of exon 3 of human tetranectin (SEQ ID NO: 59); murine tetranectin (SEQ ID NO: 60); tetranectin homologous protein isolated from reefshark cartilage (SEQ ID NO: 61) and tetranectin homologous protein isolated from bovine cartilage (SEQ ID NO: 62. Residues at a and d positions in the heptad repeats are listed in boldface. The listed consensus sequence of the tetranectin protein family trimerising structural element comprise the residues present at a and d positions in the heptad repeats shown in the figure in addition to the other conserved residues of the region. “hy” denotes an aliphatic hydrophobic residue.

FIG. 2 shows the results of CII-H6-GrB-TripK-IL-1Ra refolding by dialysis.

FIG. 3 displays the capturing CII-H6-GrB-TripK-IL-1Ra on NiNTA.

FIG. 4 is a graph showing the ability of GG-TripV-IL-1Ra (trip V-IL-1Ra), GG-TripK-IL-1Ra (trip K-IL-1Ra), GG-TripT-IL-1Ra (trip T-IL-1Ra) and GG-TripT-IL-1Ra (trip T-IL-1Ra) to inhibit IL-1 induction of IL-8 in U937 cells.

FIG. 5 is a graph showing the ability of pegylated TripT and TripV to inhibit IL-1 induction of IL-8 in U937 cells as compared to non-pegylated forms and KINERET®.

FIG. 6 is a graph showing the ability of TripT-IL-1Ra, I10-TripT-IL-1Ra, V17-TripT-IL-1Ra used in the PK study to inhibit IL-1 induction of IL-8 in U937 cells

FIG. 7 is a graph showing the blood concentrations of TripT-IL-1Ra, I10-TripT-IL-1Ra, and V17-TripT-IL-1Ra after 100 mg/kg i.v. injection in rats.

FIG. 8 shows an SDS-PAGE analysis of multiple batches of Met-1,0-TrpT-IL-1Ra (LM022 and LM023) and GG-V17-TrpT-IL-1Ra (CF019 and CF020) protein yields.

FIG. 9 shows analytical SEC results of Met-I10-TrpT-IL-1Ra and GG-V17-TrpT-IL-1Ra protein yields.

FIG. 10 shows the results of the rat CIA study. Ankle diameters of female Lewis rats with type II collagen arthritis were measured following treatment with Vehicle (10 mM phosphate buffer pH 7.4), or equimolar amounts of IL-1ra administering either monomeric IL-1ra (100 mg/kg KINERET®), or trimerized IL1ra (120 mg/kg Met-1,0-TripT-IL1ra, or 120 mg/kg GG-V17-TripT-IL1ra).

FIG. 11 shows study reduction of final paw weight when rats treated with KINERET®, Met-I10-TripT-IL1ra QD, or GG-V17-TripT-IL1ra QD, as compared to vehicle treated disease control animals.

FIG. 12 shows reduction of blood glucose levels observed after daily i.p. dosing of either I10-TripT-IL1-Ra or KINERET®.

### SUMMARY OF THE INVENTION

The present invention provides a fusion protein comprising a trimerizing domain and an IL-1Ra polypeptide sequence that inhibits IL-1 activity. In one embodiment, the fusion protein comprises an IL-1Ra sequence that comprises a variant or fragment of SEQ ID NO: 38 that inhibits IL-1 activity. In an additional embodiment, the fusion protein comprises an IL-1Ra polypeptide sequence that is at least 85% identical to SEQ ID NO: 38. The fusion proteins may include polyethylene glycol. The trimerizing domain of the fusion protein may be derived from tetranectin.

The present invention also provides a trimeric complex comprising three fusion proteins of the invention. In one embodiment, the trimeric complex comprises a trimerizing domain that is a tetranectin trimerizing structural element (TTSE). In one embodiment, the trimeric complex comprises a trimerizing domain is at least 66% identical to SEQ ID NO: 1. In further embodiment, the trimeric complex comprises at least one of the fusion proteins selected from the group consisting of TripK-IL-1ra (SEQ ID NO: 39); TripV-IL-1ra (SEQ ID NO: 40); TripT-IL-1ra (SEQ ID NO: 41); TripQ-IL-1ra (SEQ ID NO: 42); I10-TripK-IL-1ra (SEQ ID NO: 43); I10-TripV-IL-1ra (SEQ ID NO: 44); I10-TripT-IL-1ra (SEQ ID NO: 45); I10-TripQ-IL-1ra (SEQ ID NO: 46); V17-TripT-IL1Ra (SEQ ID NO: 55); V17-TripK-IL-1Ra (SEQ ID NO: 56); V17-TripV-IL-1RA (SEQ ID NO: 57); and V17-TripQ-IL1RA (SEQ ID NO: 58).

In a further embodiment, the present invention provides a pharmaceutical composition comprising a trimeric and at least one pharmaceutically acceptable excipient.

Even further, the invention is directed to a method for treating a disease mediated by interleukin 1. The method includes administering to a patient in need thereof of the pharmaceutical composition of the invention. The disease may be an inflammatory disease such as rheumatoid arthritis or diabetes. The method also includes administering to the patient, either simultaneously or sequentially, an anti-inflammatory agent.

The invention also provides a fusion protein further comprising an anti-inflammatory agent covalently linked to the fusion protein.

These and other aspects of the invention are described in further detail below.

### DETAILED DESCRIPTION OF THE INVENTION

The invention is directed to compounds and methods for treating diseases mediated by IL-1. In one aspect, the invention is directed to a fusion protein of an IL-1Ra polypeptide sequence fused to a trimerizing or multimerizing domain. Three or more of fusion proteins may trimerize or multimerize to provide compositions providing for greater stability and improved pharmacokinetic properties than IL-1Ra alone, and provide a favorable safety profile.

In an additional aspect the invention provides a nucleic acid which encodes any one of the polypeptides defined above, as well as methods of preparing these polypeptides under conditions that allow for specific expression and recovery.

The polypeptides of the invention may be used for the preparation of pharmaceutical compositions for use in the treatment of a subject having a pathology mediated by IL-1, such as a method of treatment of inflammatory diseases, by administering to the subject an effective amount of pharmaceutical composition.

As used herein, a disease or medical condition is considered to be an “interleukin-1 mediated disease” or “a disease mediated by interleukin-1” if the spontaneous or experimental disease or medical condition is associated with elevated levels of IL-1 in bodily fluids or tissue or if cells or tissues taken from the body produce elevated levels of IL-1 in culture. In many cases, such interleukin-1 mediated diseases are also recognized by the following additional two conditions: (1) pathological findings associated with the disease or medical condition can be mimicked experimentally in animals by the administration of IL-1; and (2) the pathology induced in experimental animal models of the disease or medical condition can be inhibited or abolished by treatment with agents which inhibit the action of IL-1. In most IL-1 mediated diseases at least two of the three conditions are met, and in many IL-1 mediated diseases all three conditions are met. A non-exclusive list of acute and chronic IL-1-mediated inflammatory diseases includes but is not limited to the following: gout, acute pancreatitis; ALS; Alzheimer's disease; cachexia/anorexia; asthma; atherosclerosis; chronic fatigue syndrome, fever; diabetes (e.g., insulin diabetes); glomerulonephritis; graft versus host rejection; hemohorragic shock; hyperalgesia, inflammatory bowel disease; inflammatory conditions of a joint, including osteoarthritis, psoriatic arthritis, juvenile arthritis, and rheumatoid arthritis; ischemic injury, including cerebral ischemia (e.g., brain injury as a result of trauma, epilepsy, hemorrhage or stroke, each of which may lead to neurodegeneration); lung diseases (e.g., ARDS); multiple myeloma; multiple sclerosis; myelogenous (e.g., AML and CML) and other leukemias; myopathies (e.g., muscle protein metabolism, esp. in sepsis); osteoporosis; Parkinson's disease; pain; pre-term labor; psoriasis; reperfusion injury; septic shock; side effects from radiation therapy, temporal mandibular joint disease, tumor metastasis; or an inflammatory condition resulting from strain, sprain, cartilage damage, trauma, orthopedic surgery, infection or other disease processes, and Cryopyrin-associated periodic syndromes, including Muckle Wells syndrome, familial cold autoinflammatory syndrome and neonatal-onset multisystem inflammatory disease.

As used herein, the term “multimerizing domain” means an amino acid sequence that comprises the functionality that can associate with two or more other amino acid sequences to form trimers or other multimerized complexes. In one example, the fusion protein contains an amino acid sequence—a trimerizing domain—which forms a trimeric complex with two other trimerizing domains. A trimerizing domain can associate with other trimerizing domains of identical amino acid sequence (a homotrimer), or with trimerizing domains of different amino acid sequence (a heterotrimer). Such an interaction may be caused by covalent bonds between the components of the trimerizing domains as well as by hydrogen bond forces, hydrophobic forces, van der Waals forces and salt bridges. In various embodiment so of the invention, the multimerizing domain is a dimerizing, domain, a trimerizing domain, a tetramerizing domain, a pentamerizing domain, etc. These domains are capaple of forming polypeptide complexes of two, three, four, five or more fusion proteins of the invention.

The trimerizing domain of a fusion protein of the invention may be derived from tetranectin as described in U.S. Patent Application Publication No. 2007/0154901 ('901 application), which is incorporated by reference in its entirety. The full length human tetranectin polypeptide sequence is provided herein as SEQ ID NO: 63. Examples of a tetranectin trimerizing domain includes the amino acids 17 to 49, 17 to 50, 17 to 51 and 17-52 of SEQ ID NO: 63, which represent the amino acids encoded by exon 2 of the human tetranectin gene, and optionally the first one, two or three amino acids encoded by exon 3 of the gene. Other examples include amino acids 1 to 49, 1 to 50, 1 to 51 and 1 to 52, which represents all of exons 1 and 2, and optionally the first one, two or three amino acids encoded by exon 3 of the gene. Alternatively, only a part of the amino acid sequence encoded by exon 1 is included in the trimerizing domain. In particular, the N-terminus of the trimerizing domain may begin at any of residues 1, 2, 3, 4, 5, 6, 7, 8, 9, 10, 11, 12, 13, 14, 15, 16 and 17 of SEQ ID NO: 63. In particular embodiments, the N terminus is I10 or V17 and the C-terminus is Q47, T48, V49, C(S)50, L51 or K52 (numbering according to SEQ ID NO: 63).

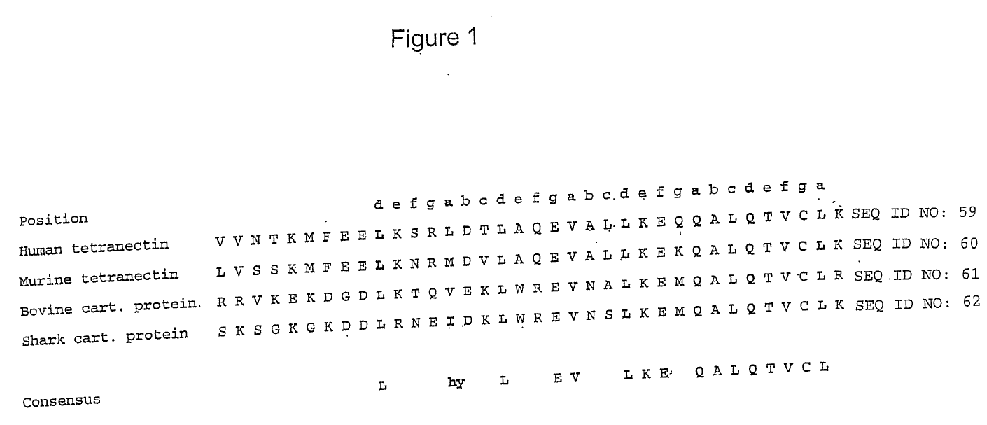

In one aspect of the invention, the trimerizing domain is a tetranectin trimerizing structural element (“TTSE”) having a amino acid sequence of SEQ ID NO: 1 which a consensus sequence of the tetranectin family trimerizing structural element as more fully described in US 2007/00154901. As shown in FIG. 1, the TTSE embraces variants of a naturally occurring member of the tetranectin family of proteins, and in particular variants that have been modified in the amino acid sequence without adversely affecting, to any substantial degree, the ability of the TTSE to form alpha helical coiled coil trimers. In various aspects of the invention, the trimeric polypeptide according to the invention includes a TTSE as a trimerizing domain having at least 66% amino acid sequence identity to the consensus sequence of SEQ ID NO: 1; for example at least 73%, at least 80%, at least 86% or at least 92% sequence identity to the consensus sequence of SEQ ID NO: 1 (counting only the defined (not Xaa) residues). In other words, at least one, at least two, at least three, at least four, or at least five of the defined amino acids in SEQ ID NO: 1 may be substituted.

In one particular embodiment, the cysteine at position 50 (C50) of SEQ ID NO: 63 can be advantageously be mutagenized to serine, threonine, methionine or to any other amino acid residue in order to avoid formation of an unwanted inter-chain disulphide bridge, which can lead to unwanted multimerization. Other known variants include at least one amino acid residue selected from amino acid residue nos. 6, 21, 22, 24, 25, 27, 28, 31, 32, 35, 39, 41, and 42 (numbering according to SEQ ID NO:63), which may be substituted by any non-helix breaking amino acid residue. These residues have been shown not to be directly involved in the intermolecular interactions that stabilize the trimeric complex between three TTSEs of native tetranectin monomers. In one aspect shown in FIG. 1, the TTSE has a repeated heptad having the formula a-b-c-d-e-f-g (N to C), wherein residues a and d (i.e., positions 26, 33, 37, 40, 44, 47, and 51 may be any hydrophobic amino acid (numbering according to claim 63).

In further embodiments, the TTSE trimerization domain may be modified by the incorporation of polyhistidine sequence and/or a protease cleavage site, e.g, Blood Coagulating Factor Xa or Granzyme B (see US 2005/0199251, which is incorporated herein by reference), and by including a C-terminal KG or KGS sequence. Also, to assist in purification, Proline at position 2 may be substituted with Glycine to assist in purification.

Particular non-limiting examples of TTSE truncations and variants are shown in Table 1 below.

TABLE 1

TTSE variants

| | |
|---|---|
| SEQ ID NO: 2 | EPPTQKPKKIVNAKKDVVNTKMFEELKSRLDTLAQEVALLKEQQALQTVSLK |
| SEQ ID NO: 3 | EPPTQKPKKIVNAKKDVVNTKMFEELKSRLDTLAQEVALLKEQQALQTVSL |
| SEQ ID NO: 4 | EPPTQKPKKIVNAKKDVVNTKMFEELKSRLDTLAQEVALLKEQQALQTVS |
| SEQ ID NO: 5 | EPPTQKPKKIVNAKKDVVNTKMFEELKSRLDTLAQEVALLKEQQALQTV |
| SEQ ID NO: 6 | PPTQKPKKIVNAKKDVVNTKMFEELKSRLDTLAQEVALLKEQQALQTVSLKG |
| SEQ ID NO: 7 | PTQKPKKIVNAKKDVVNTKMFEELKSRLDTLAQEVALLKEQQALQTVSLKG |
| SEQ ID NO: 8 | TQKPKKIVNAKKDVVNTKMFEELKSRLDTLAQEVALLKEQQALQTVSLKG |
| SEQ ID NO: 9 | QKPKKIVNAKKDVVNTKMFEELKSRLDTLAQEVALLKEQQALQTVSLKG |
| SEQ ID NO: 10 | KPKKIVNAKKDVVNTKMFEELKSRLDTLAQEVALLKEQQALQTVSLKG |
| SEQ ID NO: 11 | PKKIVNAKKDVVNTKMFEELKSRLDTLAQEVALLKEQQALQTVSLKG |
| SEQ ID NO: 12 | KKIVNAKKDVVNTKMFEELKSRLDTLAQEVALLKEQQALQTVSLKG |
| SEQ ID NO: 13 | KIVNAKKDVVNTKMFEELKSRLDTLAQEVALLKEQQALQTVSLKG |
| SEQ ID NO: 14 | IVNAKKDVVNTKMFEELKSRLDTLAQEVALLKEQQALQTVSLKG |
| SEQ ID NO: 15 | VNAKKDVVNTKMFEELKSRLDTLAQEVALLKEQQALQTVSLKG |
| SEQ ID NO: 16 | NAKKDVVNTKMFEELKSRLDTLAQEVALLKEQQALQTVSLKG |
| SEQ ID NO: 17 | AKKDVVNTKMFEELKSRLDTLAQEVALLKEQQALQTVSLKG |
| SEQ ID NO: 18 | KKDVVNTKMFEELKSRLDTLAQEVALLKEQQALQTVSLKG |
| SEQ ID NO: 19 | KDXVNTKMFEELKSRLDTLAQEVALLKEQQALQTVSLKG |
| SEQ ID NO: 20 | VVNTKMFEELKSRLDTLAQEVALLKEQQALQTVSLKG |
| SEQ ID NO: 21 | VVNTKMFEELKSRLDTLAQEVALLKEQQALQTVSLKG |
| SEQ ID NO: 22 | VVNTKMFEELKSRLDTLAQEVALLKEQQALQTVSLK |
| SEQ ID NO: 23 | VVNTKMFEELKSRLDTLAQEVALLKEQQALQTV |
| SEQ ID NO: 24 | VVNTKMFEELKSRLDTLAQEVALLKEQQALQT |
| SEQ ID NO: 25 | VNTKMFEELKSRLDTLAQEVALLKEQQALQ |
| SEQ ID NO: 26 | NTKMFEELKSRLDTLAQEVALLKEQQALQTVSLKG |
| SEQ ID NO: 27 | TKMFEELKSRLDTLAQEVALLKEQQALQTVSLKG |
| SEQ ID NO: 28 | KMFEELKSRLDTLAQEVALLKEQQALQTVSLKG |
| SEQ ID NO: 29 | MFEELKSRLDTLAQEVALLKEQQALQTVSLKG |
| SEQ ID NO: 30 | EGPTQKPKKIVNAKKDVVNTKMFEELKSRLDTLAQEVALLKEQQALQTVSLK |
| SEQ ID NO: 31 | EGPTQKPKKIVNAKKDVVNTKMFEELKSRLDTLAQEVALLKEQQALQTV |
| SEQ ID NO: 32 | EGPTQKPKKIVNAKKDVVNTKMFEELKSRLDTLAQEVALLKEQQALQT |
| SEQ ID NO: 33 | EGPTQKPKKIVNAKKDVVNTKMFEELKSRLDTLAQEVALLKEQQALQ |
| SEQ ID NO: 34 | IVNAKKDVVNTKMFEELKSRLDTLAQEVALLKEQQALQTVSLK |
| SEQ ID NO: 35 | IVNAKKDVVNTKMFEELKSRLDTLAQEVALLKEQQALQTV |
| SEQ ID NO: 36 | IVNAKKDVVNTKMFEELKSRLDTLAQEVALLKEQQALQT |
| SEQ ID NO: 37 | IVNAKKDVVNTKMFEELKSRLDTLAQEVALLKEQQALQ |

Another example of a trimerizing domain is disclosed in U.S. Pat. No. 6,190,886 (incorporated herein in its entirety), which describes polypeptides comprising a collectin neck region. Trimers can then be made under appropriate conditions with three polypeptides comprising the collectin neck region amino acid sequence.

Another example of a trimerizing domain is an MBP trimerizing domain, as described in U.S. Provisional Patent Application Ser. No. 60/996,288, filed by the assignee of the present application on Nov. 9, 2007, which is incorporated by reference in its entirety. This trimerizing domain can oligomerize even further and create higher order multimeric complexes.

The IL-1 Ra polypeptide of the invention may either be linked to the N- or the C-terminal amino acid residue of the trimerization domain. A flexible molecular linker optionally may be interposed between, and covalently join, the polypeptide representing the IL-1 Ra and the trimerization domain. Preferably, the linker is a polypeptide sequence of about 1 to 20, 2 to 10, or 3 to 7 amino acid residues. In further embodiments, the linker is non-immunogenic, not prone to proteolytic cleavage, and does not comprise amino acid residues which are known to interact with other residues (e.g. cystein residues).

As used herein “IL-1Ra” refers to a polypeptide having the amino acid sequence shown below:

(SEQ ID NO: 38)

RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE

Also included in the “IL-1Ra” definition are variants and fragments of SEQ ID NO: 38 that provide for IL-1Ra binding to IL-1R, and preferably IL-1R inhibitory activity. Such fragments may be truncated at the N-terminus or C-terminus of the IL-1Ra, or may lack internal residues, when compared with the full length native IL-1Ra protein. Certain fragments may lack amino acid residues that are not essential for a desired biological activity of the trimeric IL-1Ra protein according to the invention. For example, Evans, et al. (J. Biol. Chem. 1995, 19:11477-11483) demonstrated by site directed mutagenesis that only Trp16, Gln20, Tyr34, Gln36 and Tyr147 are critical for binding to the IL-1R and that other amino acid positions can be altered while still maintaining a functional molecule. Furthermore, affinity of IL-1Ra to its receptor can be improved by mutating amino acids outside the binding region to increase loop interactions of IL-1Ra with its receptor as shown by Dahlen, et al, (J. Immunotoxicology 5:189-199 (2008)). This is can be accomplished through mutations of amino acids outside the IL-1Ra receptor binding region, and particularly, for example: D47N, E52R, E90Y, P38Y, H54R, Q129L and M136N. Id. Furthermore, natural IL-1Ra variants exist, any of which may be used. An 18 kDa form of IL-1Ra, created by an alternative transcriptional splice mechanism from an upstream exon is called icIL-1Ra1 and is found inside keratinocytes and other epithelial cells, monocytes, tissue macrophages, fibroblasts, and endothelial cells. IL-1Ra cDNA cloned from human leukocytes contains an additional 63 bp sequence as an insert in the 5′ region of the cDNA. A 15 kDa isoform of IL-1Ra, termed icIL-1Ra3, is found in monocytes, macrophages, neutrophils, and hepatocytes, and may be created both by an alternative transcriptional splice as well as by alternative translational initiation.

IL-1Ra peptides that are useful for fusion proteins of the invention include polypeptides that are at least 65%, at least 70%, at least 75%, at least 80%, at least 85%, at least 90%, or at least 95% identical to SEQ ID NO: 38. In particular embodiments, the fusion proteins include an IL-1Ra peptide sequence that is 85% identical to SEQ ID NO: 38 and has IL-1R binding activity, and preferably IL-1Ra inhibitory activity. In another particular embodiment, the fusion proteins include an IL-1Ra peptide sequence that is 95% identical to SEQ ID NO: 38 and has IL-1R binding activity, and preferably IL-1Ra inhibitory activity. In these embodiment, the polypeptides comprise Trp16, Gln20, Tyr34, Gln36 and Tyr147 according to the numbering of SEQ ID NO: 38 These polypeptides may further include one or more amino acids substitutions D47N, E52R, E90Y, P38Y, H54R, Q129L and M136N (numbering according to SEQ ID NO: 38). Furthermore, variations of the IL-1Ra polypeptides can be accomplished by replacing one or more amino acids with another amino acid having similar structural or chemical properties, for example, conservative amino acid substitutions.

In a further embodiment, the fusion protein according to the invention is selected from an IL-1 receptor antagonist selected from the following:

TripK-IL-1ra

(SEQ ID NO: 39)

EGPTQKPKKIVNAKKDVVNTKMFEELKSRLDTLAQEVALLKEQQALQTVS
LKRPSGRKSSKMQAFRIWDVNQKTFYLRNNQLVAGYLQGPNVNLEEKIDV
VPIEPHALFLGIHGGKMCLSCVKSGDETRLQLEAVNITDLSENRKQDKRF
AFIRSDSGPTTSFESAACPGWFLCTAMEADQPVSLTNMPDEGVMVTKFYF
QEDE;
TripV-IL-1ra

(SEQ ID NO: 40)

EGPTQKPKKIVNAKKDVVNTKMFEELKSRLDTLAQEVALLKEQQALQTVR
PSGRKSSKMQAFRIWDVNQKTFYLRNNQLVAGYLQGPNVNLEEKIDVVPE
PHALFLGIHGGKMCLSCVKSGDETRLQLEAVNITDLSENRKQDKRFAFIR
SDSGPTTSFESAACPGWFLCTAMEADQPVSLTNMPDEGVMVTKFYFQED
E;
TripT-IL-1ra

(SEQ ID NO: 41)

EGPTQKPKKIVNAKKDVVNTKMFEELKSRLDTLAQEVALLKEQQALQTRP
SGRKSSKMQAFRIWDVNQKTFYLRNNQLVAGYLQGPNVNLEEKIDVVPIE
PHALFLGIHGGKMCLSCVKSGDETRLQLEAVNITDLSENRKQDKRFAFIR
SDSGPTTSFESAACPGWFLCTAMEADQPVSLTNMPDEGVMVTKFYFQEDE
TripQ-IL-1ra

(SEQ ID NO: 42)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQRPSGR
KSSKMQAFRIWDVNQKTFYLRNNQLVAGYLQGPNVNLEEKIDVVPIEPHA
LFLGIHGGKMCLSCVKSGDETRLQLEAVNITDLSENRKQDKRFAFIRSDS
GPTTSFESAACPGWFLCTAMEADQPVSLTNMPDEGVMVTKFYFQEDE
I10-TripK-IL1ra

(SEQ ID NO: 43)

IVNAKKDVVNTKMFEELKSRLDTLAQEVALLKEQQALQTVSLKRPSGRKS
SKMQAFRIWDVNQKTFYLRNNQLVAGYLQGPNVNLEEKIDVVPIEPHALF
LGIHGGKMCLSCVKSGDETRLQLEAVNITDLSENRKQDKRFAFIRSDSGP
TTSFESAACPGWFLCTAMEADQPVSLTNMPDEGVMVTKFYFQEDE;
I10-TripV-IL-1ra

(SEQ ID NO: 44)

IVNAKKDVVNTKMFEELKSRLDTLAQEVALLKEQQALQTVRPSGRKSSKM
QAFRIWDVNQKTFYLRNNQLVAGYLQGPNVNLEEKIDVVPIEPHALFLGI
HGGKMCLSCVKSGDETRLQLEAVNITDLSENRKQDKRFAFIRSDSGPTTS
FESAACPGWFLCTAMEADQPVSLTNMPDEGVMVTKFYFQEDE;
I10-TripT-IL-1ra

(SEQ ID NO: 45)

IVNAKKDVVNTKMFEELKSRLDTLAQEVALLKEQQALQTRPSGRKSSKMQ
AFRIWDVNQKTFYLRNNQLVAGYLQGPNVNLEEKIDVVPIEPHALFLGIH
GGKMCLSCVKSGDETRLQLEAVNITDLSENRKQDKRFAFIRSDSGPTTSF
ESAACPGWFLCTAMEADQPVSLTNMPDEGVMVTKFYFQEDE;
I10-TripQ-IL-1ra

(SEQ ID NO: 46)

IVNAKKDVVNTKMFEELKSRLDTLAQEVALLKEQQALQRPSGRKSSKMQA
FRIWDVNQKTFYLRNNQLVAGYLQGPNVNLEEKIDVVPIEPHALFLGIHG
GKMCLSCVKSGDETRLQLEAVNITDLSENRKQDKRFAFIRSDSGPTTSFE
SAACPGWFLCTAMEADQPVSLTNMPDEGVMVTKFYFQEDE;

wherein the underlined part denotes the trimerization unit, and the bold part denotes the IL-1Ra part.

### Production of Fusion Proteins

The trimeric IL-1Ra protein of the invention may be chemically synthesized or expressed in any suitable standard protein expression system. Preferably, the protein expression systems are systems from which the desired protein may readily be isolated and refolded in vitro. Prokaryotic expression systems are preferred since high yields of protein can be obtained and efficient purification and refolding strategies are available. Eukaryotic expression systems may also be used. Thus, it is well within the abilities and discretion of the skilled artisan to choose an appropriate expression system. Similarly, once the primary amino acid sequence for the fusion proteins of the present invention is chosen, one of ordinary skill in the art can easily design appropriate recombinant DNA constructs which will encode the desired proteins, taking into consideration such factors as codon biases in the chosen host, the need for secretion signal sequences in the host, the introduction of proteinase cleavage sites within the signal sequence, and the like. These recombinant DNA constructs may be inserted in-frame into any of a number of expression vectors appropriate to the chosen host. Preferably, the expression vector will include a strong promoter to drive expression of the recombinant constructs.

The fusion protein of the invention can be expressed in any suitable standard protein expression system by culturing a host transformed with a vector encoding the fusion protein under such conditions that the fusion protein is expressed. Preferably, the expression system is a system from which the desired protein may readily be isolated and refolded in vitro. As a general matter, prokaryotic expression systems are preferred since high yields of protein can be obtained and efficient purification and refolding strategies are available. Thus, selection of appropriate expression systems (including vectors and cell types) is within the knowledge of one skilled in the art. Similarly, once the primary amino acid sequence for the fusion protein of the present invention is chosen, one of ordinary skill in the art can easily design appropriate recombinant DNA constructs which will encode the desired amino acid sequence, taking into consideration such factors as codon biases in the chosen host, the need for secretion signal sequences in the host, the introduction of proteinase cleavage sites within the signal sequence, and the like.

In one embodiment the isolated polynucleotide encodes a fusion protein of the invention. In other embodiments, an IL-1Ra polypeptide and the trimerizing domain are encoded by non-contiguous polynucleotide sequences. Accordingly, in some embodiments an IL-1Ra polypeptide and the trimerizing domain are expressed, isolated, and purified as separate polypeptides and fused together to form the fusion protein of the invention.

These recombinant DNA constructs may be inserted in-frame into any of a number of expression vectors appropriate to the chosen host. In certain embodiments, the expression vector comprises a strong promoter that controls expression of the recombinant fusion protein constructs. When recombinant expression strategies are used to generate the fusion protein of the invention, the resulting fusion protein can be isolated and purified using suitable standard procedures well known in the art, and optionally subjected to further processing such as e.g. lyophilization.

Standard techniques may be used for recombinant DNA molecule, protein, and fusion protein production, as well as for tissue culture and cell transformation. See, e.g., Sambrook, et al. (below) or Current Protocols in Molecular Biology (Ausubel et al., eds., Green Publishers Inc. and Wiley and Sons 1994). Purification techniques are typically performed according to the manufacturer's specifications or as commonly accomplished in the art using conventional procedures such as those set forth in Sambrook et al. (Molecular Cloning: A Laboratory Manual. Cold Spring Harbor Laboratory Press, Cold Spring Harbor, N.Y. (1989), or as described herein. Unless specific definitions are provided, the nomenclature utilized in connection with the laboratory procedures, and techniques relating to molecular biology, biochemistry, analytical chemistry, and pharmaceutical/formulation chemistry described herein are those well known and commonly used in the art. Standard techniques can be used for biochemical syntheses, biochemical analyses, pharmaceutical preparation, formulation, and delivery, and treatment of patients.

It will be appreciated that a flexible molecular linker optionally may be interposed between, and covalently join, the IL-1Ra polypeptide and the trimerizing domain. In certain embodiments, the linker is a polypeptide sequence of about 1 to 20 amino acid residues. The linker may be less than 10 amino acids, most preferably, five, four, three, two, or one amino acid. It may be in certain cases that nine, eight, seven, or six amino acids are suitable. In useful embodiments the linker is essentially non-immunogenic, not prone to proteolytic cleavage and does not comprise amino acid residues which are known to interact with other residues (e.g. cysteine residues).

The description below also relates to methods of producing fusion proteins and trimeric complexes that are covalently attached (hereinafter “conjugated”) to one or more chemical groups. Chemical groups suitable for use in such conjugates are preferably not significantly toxic or immunogenic. The chemical group is optionally selected to produce a conjugate that can be stored and used under conditions suitable for storage. A variety of exemplary chemical groups that can be conjugated to polypeptides are known in the art and include for example carbohydrates, such as those carbohydrates that occur naturally on glycoproteins, polyglutamate, and non-proteinaceous polymers, such as polyols (see, e.g., U.S. Pat. No. 6,245,901).

A polyol, for example, can be conjugated to fusion proteins of the invention at one or more amino acid residues, including lysine residues, as is disclosed in WO 93/00109, supra. The polyol employed can be any water-soluble poly(alkylene oxide) polymer and can have a linear or branched chain. Suitable polyols include those substituted at one or more hydroxyl positions with a chemical group, such as an alkyl group having between one and four carbons. Typically, the polyol is a poly(alkylene glycol), such as poly(ethylene glycol) (PEG), and thus, for ease of description, the remainder of the discussion relates to an exemplary embodiment wherein the polyol employed is PEG and the process of conjugating the polyol to a polypeptide is termed “pegylation.” However, those skilled in the art recognize that other polyols, such as, for example, poly(propylene glycol) and polyethylene-polypropylene glycol copolymers, can be employed using the techniques for conjugation described herein for PEG.

The average molecular weight of the PEG employed in the pegylation of IL-1Ra can vary, and typically may range from about 500 to about 30,000 daltons (D). Preferably, the average molecular weight of the PEG is from about 1,000 to about 25,000 D, and more preferably from about 1,000 to about 5,000 D. In one embodiment, pegylation is carried out with PEG having an average molecular weight of about 1,000 D. Optionally, the PEG homopolymer is unsubstituted, but it may also be substituted at one end with an alkyl group. Preferably, the alkyl group is a C1-C4 alkyl group, and most preferably a methyl group. PEG preparations are commercially available, and typically, those PEG preparations suitable for use in the present invention are non-homogeneous preparations sold according to average molecular weight. For example, commercially available PEG (5000) preparations typically contain molecules that vary slightly in molecular weight, usually ±500 D. The fusion protein of the invention can be further modified using techniques known in the art, such as, conjugated to a small molecule compounds (e.g., a chemotherapeutic); conjugated to a signal molecule (e.g., a fluorophore); conjugated to a molecule of a specific binding pair (e.g., biotin/streptavidin, antibody/antigen); or stabilized by glycosylation, PEGylation, or further fusions to a stabilizing domain (e.g., Fc domains).

A variety of methods for pegylating proteins are known in the art. Specific methods of producing proteins conjugated to PEG include the methods described in U.S. Pat. Nos. 4,179,337, 4,935,465 and 5,849,535. Typically the protein is covalently bonded via one or more of the amino acid residues of the protein to a terminal reactive group on the polymer, depending mainly on the reaction conditions, the molecular weight of the polymer, etc. The polymer with the reactive group(s) is designated herein as activated polymer. The reactive group selectively reacts with free amino or other reactive groups on the protein. The PEG polymer can be coupled to the amino or other reactive group on the protein in either a random or a site specific manner. It will be understood, however, that the type and amount of the reactive group chosen, as well as the type of polymer employed, to obtain optimum results, will depend on the particular protein or protein variant employed to avoid having the reactive group react with too many particularly active groups on the protein. As this may not be possible to avoid completely, it is recommended that generally from about 0.1 to 1000 moles, preferably 2 to 200 moles, of activated polymer per mole of protein, depending on protein concentration, is employed. The final amount of activated polymer per mole of protein is a balance to maintain optimum activity, while at the same time optimizing, if possible, the circulatory half-life of the protein.

The term “polyol” when used herein refers broadly to polyhydric alcohol compounds. Polyols can be any water-soluble poly(alkylene oxide) polymer for example, and can have a linear or branched chain. Preferred polyols include those substituted at one or more hydroxyl positions with a chemical group, such as an alkyl group having between one and four carbons. Typically, the polyol is a poly(alkylene glycol), preferably poly(ethylene glycol) (PEG). However, those skilled in the art recognize that other polyols, such as, for example, poly(propylene glycol) and polyethylene-polypropylene glycol copolymers, can be employed using the techniques for conjugation described herein for PEG. The polyols of the invention include those well known in the art and those publicly available, such as from commercially available sources.

Furthermore, other half-life extending molecules can be attached to the N- or C-terminus of the trimerization domain including serum albumin-binding peptides, FcRn-binding peptides or IgG-binding peptides.

In one embodiment, the trimeric IL-1Ra protein of the invention is expressed in a prokaryotic host cell such as E. coli and is additionally linked to a third polypeptide, i.e. a third fusion partner. Thus, it may be that by adding such third fusion partner to the trimeric IL-1Ra protein of the invention, high yields of the trimeric IL-1Ra protein may be obtained. The third fusion partner may be any suitable peptide, oligopeptide, polypeptide or protein, including a di-peptide, a tri-peptide, tetra-peptide, penta-peptide or hexa-peptide. The fusion partner may in certain instances be a single amino acid. It may be selected such that it renders the fusion protein more resistant to proteolytic degradation, facilitates enhanced expression and secretion of the fusion protein, improves solubility, and/or allows for subsequent affinity purification of the fusion protein.

In one embodiment, the junction region between the fusion protein of the invention (i.e. the IL-1Ra portion and the trimerization domain) and the third fusion partner such as ubiquitin, comprises a Granzyme B protease cleavage site such as human Granzyme B (E.C. 3.4.21.79) as described in US 2005/0199251.

The third fusion partner may in further embodiments be coupled to an affinity-tag. Such an affinity-tag may be an affinity domain which allows for the purification of the fusion protein on an affinity resin. The affinity-tag may be a polyhistidine-tag such as a hexahis-tag, polyarginine-tag, FLAG-tag, Strep-tag, c-myc-tag, S-tag, calmodulin-binding peptide, cellulose-binding peptide, chitin-binding domain, glutathione S-transferase-tag, or maltose binding protein.

The method of the invention may be in an isolation step for isolating the trimeric IL-1 Ra protein that is formed by the enzymatic cleavage of the fusion protein that has been immobilized by the use of the above mentioned affinity-tag systems. This isolation step can be performed by any suitable means known in the art for protein isolation, including the use of ion exchange and fractionation by size, the choice of which depends on the character of the fusion protein. In one embodiment, the region between the third fusion partner and the region comprising the trimerization domain and IL-1Ra is contacted with the human serine protease Granzyme B to cleave off the fusion protein at a Granzyme B protease cleavage site which yields the fusion protein of the invention.

The present invention also provides plasmids, vectors, transcription or expression cassettes which comprise at least one nucleic acid as described above. Suitable vectors can be chosen or constructed containing appropriate regulatory sequences, including promoter sequences, terminator sequences, polyadenylation sequences, enhancer sequences, marker genes and other sequences as appropriate. Vectors may be plasmids, viral, phage, or phagemid, as appropriate. (Molecular Cloning: a Laboratory Manual: 2nd edition, Sambrook et al., 1989, Cold Spring Harbor Laboratory Press).

The present invention also provides a recombinant host cell which comprises one or more constructs of the invention. Suitable host cells include bacteria, mammalian cells, yeast and baculovirus systems. Mammalian cell lines available for expression of a heterologous polypeptide include Chinese hamster ovary cells, HeLa cells, baby hamster kidney cells, NSO mouse melanoma cells and many others. A preferred bacterial host is E. coli.

Pharmaceutical Compositions

In yet another aspect, the invention relates to a pharmaceutical composition comprising a therapeutically effective amount of the fusion protein of the invention along with a pharmaceutically acceptable carrier or excipient. As used herein, “pharmaceutically acceptable carrier” or “pharmaceutically acceptable excipient” includes any and all solvents, dispersion media, coating, antibacterial and antifungal agents, isotonic and absorption delaying agents, and the like that are physiologically compatible. Examples of pharmaceutically acceptable carriers or excipients include one or more of water, saline, phosphate buffered saline, dextrose, glycerol, ethanol and the like as well as combinations thereof. In many cases, it will be preferable to include isotonic agents, for example, sugars, polyalcohols such as mannitol, sorbitol, or sodium chloride in the composition. Pharmaceutically acceptable substances such as wetting or minor amounts of auxiliary substances such as wetting or emulsifying agents, preservatives or buffers, which enhance the shelf life or effectiveness of the of the antibody or antibody portion also may be included. Optionally, disintegrating agents can be included, such as cross-linked polyvinyl pyrrolidone, agar, alginic acid or a salt thereof, such as sodium alginate and the like. In addition to the excipients, the pharmaceutical composition can include one or more of the following, carrier proteins such as serum albumin, buffers, binding agents, sweeteners and other flavoring agents; coloring agents and polyethylene glycol.

The compositions can be in a variety of forms including, for example, liquid, semi-solid and solid dosage forms, such as liquid solutions (e.g. injectable and infusible solutions), dispersions or suspensions, tablets, pills, powders, liposomes and suppositories. The preferred form will depend on the intended route of administration and therapeutic application. In an embodiment the compositions are in the form of injectable or infusible solutions, such as compositions similar to those used for passive immunization of humans with antibodies. In an embodiment the mode of administration is parenteral (e.g., intravenous, subcutaneous, intraperitoneal, intramuscular). In an embodiment, the fusion protein (or trimeric complex) is administered by intravenous infusion or injection. In another embodiment, the fusion protein or trimeric complex is administered by intramuscular or subcutaneous injection.

Other suitable routes of administration for the pharmaceutical composition include, but are not limited to, oral, rectal, transdermal, vaginal, transmucosal or intestinal administration.

Therapeutic compositions are typically sterile and stable under the conditions of manufacture and storage. The composition can be formulated as a solution, microemulsion, dispersion, liposome, or other ordered structure suitable to high drug concentration. Sterile injectable solutions can be prepared by incorporating the active compound (i.e. fusion protein or trimeric complex) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, the preferred methods of preparation are vacuum drying and freeze-drying that yields a powder of the active ingredient plus any additional desired ingredient from a previously sterile-filtered solution thereof. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

An article of manufacture such as a kit containing therapeutic agents useful in the treatment of the disorders described herein comprises at least a container and a label. Suitable containers include, for example, bottles, vials, syringes, and test tubes. The containers may be formed from a variety of materials such as glass or plastic. The label on or associated with the container indicates that the formulation is used for treating the condition of choice. The article of manufacture may further comprise a container comprising a pharmaceutically-acceptable buffer, such as phosphate-buffered saline, Ringer's solution, and dextrose solution. It may further include other materials desirable from a commercial and user standpoint, including other buffers, diluents, filters, needles, syringes, and package inserts with instructions for use. The article of manufacture may also comprise a container with another active agent as described above.

Typically, an appropriate amount of a pharmaceutically-acceptable salt is used in the formulation to render the formulation isotonic. Examples of pharmaceutically-acceptable carriers include saline, Ringer's solution and dextrose solution. The pH of the formulation is preferably from about 6 to about 9, and more preferably from about 7 to about 7.5. It will be apparent to those persons skilled in the art that certain carriers may be more preferable depending upon, for instance, the route of administration and concentrations of therapeutic agent.

Therapeutic compositions can be prepared by mixing the desired molecules having the appropriate degree of purity with optional pharmaceutically acceptable carriers, excipients, or stabilizers (Remington's Pharmaceutical Sciences, 16th edition, Osol, A. ed. (1980)), in the form of lyophilized formulations, aqueous solutions or aqueous suspensions. Acceptable carriers, excipients, or stabilizers are preferably nontoxic to recipients at the dosages and concentrations employed, and include buffers such as Tris, HEPES, PIPES, phosphate, citrate, and other organic acids; antioxidants including ascorbic acid and methionine; preservatives (such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol); low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; sugars such as sucrose, mannitol, trehalose or sorbitol; salt-forming counter-ions such as sodium; and/or non-ionic surfactants such as TWEEN™, PLURONICS™ or polyethylene glycol (PEG).

Additional examples of such carriers include ion exchangers, alumina, aluminum stearate, lecithin, serum proteins, such as human serum albumin, buffer substances such as glycine, sorbic acid, potassium sorbate, partial glyceride mixtures of saturated vegetable fatty acids, water, salts, or electrolytes such as protamine sulfate, disodium hydrogen phosphate, potassium hydrogen phosphate, sodium chloride, colloidal silica, magnesium trisilicate, polyvinyl pyrrolidone, and cellulose-based substances. Carriers for topical or gel-based forms include polysaccharides such as sodium carboxymethylcellulose or methylcellulose, polyvinylpyrrolidone, polyacrylates, polyoxyethylene-polyoxypropylene-block polymers, polyethylene glycol, and wood wax alcohols. For all administrations, conventional depot forms are suitably used. Such forms include, for example, microcapsules, nano-capsules, liposomes, plasters, inhalation forms, nose sprays, sublingual tablets, and sustained-release preparations.

Formulations to be used for in vivo administration should be sterile. This is readily accomplished by filtration through sterile filtration membranes, prior to or following lyophilization and reconstitution. The formulation may be stored in lyophilized form or in solution if administered systemically. If in lyophilized form, it is typically formulated in combination with other ingredients for reconstitution with an appropriate diluent at the time for use. An example of a liquid formulation is a sterile, clear, colorless unpreserved solution filled in a single-dose vial for subcutaneous injection.

Therapeutic formulations generally are placed into a container having a sterile access port, for example, an intravenous solution bag or vial having a stopper pierceable by a hypodermic injection needle. The formulations are preferably administered as repeated intravenous (i.v.), subcutaneous (s.c.), intramuscular (i.m.) injections or infusions, or as aerosol formulations suitable for intranasal or intrapulmonary delivery (for intrapulmonary delivery see, e.g., EP 257,956).

The molecules disclosed herein can also be administered in the form of sustained-release preparations. Suitable examples of sustained-release preparations include semipermeable matrices of solid hydrophobic polymers containing the protein, which matrices are in the form of shaped articles, e.g., films, or microcapsules. Examples of sustained-release matrices include polyesters, hydrogels (e.g., poly(2-hydroxyethyl-methacrylate) as described by Langer et al., J. Biomed. Mater. Res., 15: 167-277 (1981) and Langer, Chem. Tech., 12: 98-105 (1982) or poly(vinylalcohol)), polylactides (U.S. Pat. No. 3,773,919, EP 58,481), copolymers of L-glutamic acid and gamma ethyl-L-glutamate (Sidman et al., Biopolymers, 22: 547-556 (1983)), non-degradable ethylene-vinyl acetate (Langer et al., supra), degradable lactic acid-glycolic acid copolymers such as the Lupron Depot (injectable microspheres composed of lactic acid-glycolic acid copolymer and leuprolide acetate), and poly-D-(−)-3-hydroxybutyric acid (EP 133,988).

Methods of Treatment

Another aspect the invention relates to a method of treating diseases that are mediated by IL-1Ra. The method includes treating a subject suffering from such as disease with a therapeutically effective amount of the pharmaceutical compositions of the invention.

Another aspect of the invention is directed to a combination therapy. Formulations comprising therapeutic agents are also provided by the present invention. It is believed that such formulations will be particularly suitable for storage as well as for therapeutic administration. The formulations may be prepared by known techniques. For instance, the formulations may be prepared by buffer exchange on a gel filtration column.

The pharmaceutical compositions can be administered in accord with known methods, such as intravenous administration as a bolus or by continuous infusion over a period of time, by intramuscular, intraperitoneal, intracerobrospinal, subcutaneous, intra-articular, intrasynovial, intrathecal, oral, topical, or inhalation routes. Optionally, administration may be performed through mini-pump infusion using various commercially available devices.

Effective dosages and schedules for administering the trimeric IL-1Ra may be determined empirically, and making such determinations is within the skill in the art. Single or multiple dosages may be employed. It is presently believed that an effective dosage or amount of the trimeric IL-1Ra used alone may range from about 1 μg/kg to about 100 mg/kg of body weight or more per day. Interspecies scaling of dosages can be performed in a manner known in the art, e.g., as disclosed in Mordenti, et al., Pharmaceut. Res., 8:1351 (1991).

When in vivo administration of the IL-1Ra fusion protein is employed, normal dosage amounts may vary from about 10 ng/kg to up to 100 mg/kg of mammal body weight or more per day, preferably about 1 μg/kg/day to 50 mg/kg/day, depending upon the route of administration. Guidance as to particular dosages and methods of delivery is provided in the literature (see, for example, U.S. Pat. No. 4,657,760; 5,206,344; or 5,225,212). One of skill will appreciate that different formulations will be effective for different treatment compounds and different disorders, that administration targeting one organ or tissue, for example, may necessitate delivery in a manner different from that to another organ or tissue. Those skilled in the art will understand that the dosage of the trimeric IL-1Ra that must be administered will vary depending on, for example, the mammal which will receive trimeric IL-1Ra, the route of administration, and other drugs or therapies being administered to the mammal.

The trimeric complexes and other therapeutic agents (and one or more other therapies) may be administered concurrently (simultaneously) or sequentially. In particular embodiments, a fusion protein or trimeric complex and a therapeutic agent are administered concurrently. In another embodiment, a fusion protein or trimeric complex is administered prior to administration of a therapeutic agent. In another embodiment, a therapeutic agent is administered prior to a fusion protein or trimeric complex. Following administration, treated cells in vitro can be analyzed. Where there has been in vivo treatment, a treated mammal can be monitored in various ways well known to the skilled practitioner. For instance, serum cytokine responses can be analyzed.

The IL-1Ra fusion proteins described herein may be used in combination (pre-treatment, post-treatment, or concurrent treatment) with any of one or more TNF inhibitors for the treatment or prevention of the diseases and disorders recited herein, such as but not limited to, all forms of soluble TNF receptors including Etanercept (such as ENBREL®), as well as all forms of monomeric or multimeric p75 and/or p55 TNF receptor molecules and fragments thereof; anti-human TNF antibodies, such as but not limited to, Infliximab (such as REMICADE®), and D2E7 (such as HUMIRA®), and the like. Such TNF inhibitors include compounds and proteins which block in vivo synthesis or extracellular release of TNF. In a specific embodiment, the present invention is directed to the use of an IL-17RA IL-1Ra fusion proteins in combination (pre-treatment, post-treatment, or concurrent treatment) with any of one or more of the following TNF inhibitors: TNF binding proteins (soluble TNF receptor type-I and soluble TNF receptor type-II (“sTNFRs”), as defined herein), anti-TNF antibodies, granulocyte colony stimulating factor; thalidomide; BN 50730; tenidap; E 5531; tiapafant PCA 4248; nimesulide; panavir; rolipram; RP 73401; peptide T; MDL 201,449A; (1R,3S)-Cis-1-[9-(2,6-diaminopurinyl)]-3-hydroxy-4-cyclopentene hydrochloride; (1R,3R)-trans-1-(9-(2,6-diamino)purine]-3-acetoxycyclopentane; (1R,3R)-trans-1-(9-adenyl)-3-azidocyclopentane hydrochloride and (1R,3R)-trans-1-(6-hydroxy-purin-9-yl)-3-azidocyclo-pentane. TNF binding proteins are disclosed in the art (EP 308 378, EP 422 339, GB 2 218 101, EP 393 438, WO 90/13575, EP 398 327, EP 412 486, WO 91/03553, EP 418 014, JP 127,800/1991, EP 433 900, U.S. Pat. No. 5,136,021, GB 2 246 569, EP 464 533, WO 92/01002, WO 92/13095, WO 92/16221, EP 512 528, EP 526 905, WO 93/07863, EP 568 928, WO 93/21946, WO 93/19777, EP 417 563, WO 94/06476, and PCT International Application No. PCT/US97/12244).

For example, EP 393 438 and EP 422 339 teach the amino acid and nucleic acid sequences of a soluble TNF receptor type I (also known as “sTNFR-I” or “30 kDa TNF inhibitor”) and a soluble TNF receptor type II (also known as “sTNFR-II” or “40 kDa TNF inhibitor”), collectively termed “sTNFRs”, as well as modified forms thereof (e.g., fragments, functional derivatives and variants). EP 393 438 and EP 422 339 also disclose methods for isolating the genes responsible for coding the inhibitors, cloning the gene in suitable vectors and cell types and expressing the gene to produce the inhibitors. Additionally, polyvalent forms (i.e., molecules comprising more than one active moiety) of sTNFR-1 and sTNFR-II have also been disclosed. In one embodiment, the polyvalent form may be constructed by chemically coupling at least one TNF inhibitor and another moiety with any clinically acceptable linker, for example polyethylene glycol (WO 92/16221 and WO 95/34326), by a peptide linker (Neve et al. (1996), Cytokine, 8(5):365-370, by chemically coupling to biotin and then binding to avidin (WO 91/03553) and, finally, by combining chimeric antibody molecules (U.S. Pat. No. 5,116,964, WO 89/09622, WO 91/16437 and EP 315062.

Anti-TNF antibodies include the MAK 195F Fab antibody (Holler et al. (1993), 1st International Symposium on Cytokines in Bone Marrow Transplantation, 147); CDP 571 anti-TNF monoclonal antibody (Rankin et al. (1995), British Journal of Rheumatology, 34:334-342); BAY X 1351 murine anti-tumor necrosis factor monoclonal antibody (Kieft et al. (1995), 7th European Congress of Clinical Microbiology and Infectious Diseases, page 9); CenTNF cA2 anti-TNF monoclonal antibody (Elliott et al. (1994), Lancet, 344:1125-1127 and Elliott et al. (1994), Lancet, 344:1105-1110).

The IL-1Ra fusion proteins described herein may be used in combination with all forms of IL-17 inhibitors (e.g. anti-IL17 receptor antibody, Amgen; anti-IL-17A, anti-IL17F), RORc inhibitors.

The IL-1Ra fusion proteins described herein may be used in combination with all forms of CD28 inhibitors, such as but not limited to, abatacept (for example ORENCIA®).

The IL-1Ra fusion proteins described herein may be used in combination with all forms of IL-6 and/or IL-6 receptor inhibitors, such as but not limited to, Tocilizumab (for example ACTEMRA®).

The IL-1Ra fusion proteins described herein may be used in combination with all forms of anti-IL-18 compounds, such as IL-18BP or a derivative, an IL-18 trap, anti-IL-18, anti-IL-18R1, or anti-IL-18RAcP.

The IL-1Ra fusion proteins described herein may be used in combination with all forms of anti-IL22, such as anti-IL22 or anti-IL22R.

The IL-1Ra fusion proteins described herein may be used in combination with all forms of anti-IL-23 and or IL-12 such as anti-p19, anti-p40 (Ustekinumab), anti-IL-23R.

The IL-1Ra fusion proteins described herein may be used in combination with all forms of anti-IL21, such as anti-IL21 or anti-IL21R.

The IL-1Ra fusion proteins described herein may be used in combination with all forms of anti-TGF-beta.

The IL-1Ra fusion proteins may be used in combination with one or more cytokines, lymphokines, hematopoietic factor(s), and/or an anti-inflammatory agent.

Treatment of the diseases and disorders recited herein can include the use of first line drugs for control of pain and inflammation in combination (pretreatment, post-treatment, or concurrent treatment) with treatment with one or more of the IL-1Ra fusion proteins provided herein. These drugs are classified as non-steroidal, anti-inflammatory drugs (NSAIDs). Secondary treatments include corticosteroids, slow acting antirheumatic drugs (SAARDs), or disease modifying (DM) drugs. Information regarding the following compounds can be found in The Merck Manual of Diagnosis and Therapy, Sixteenth Edition, Merck, Sharp & Dohme Research Laboratories, Merck & Co., Rahway, N.J. (1992) and in Pharmaprojects, PJB Publications Ltd.

The IL-1Ra fusion proteins described herein may be used in combination with any of one or more NSAIDs for the treatment of the diseases and disorders recited herein. NSAIDs owe their anti-inflammatory action, at least in part, to the inhibition of prostaglandin synthesis (Goodman and Gilman in “The Pharmacological Basis of Therapeutics,” MacMillan 7th Edition (1985)). NSAIDs can be characterized into at least nine groups: (1) salicylic acid derivatives; (2) propionic acid derivatives; (3) acetic acid derivatives; (4) fenamic acid derivatives; (5) carboxylic acid derivatives; (6) butyric acid derivatives; (7) oxicams; (8) pyrazoles and (9) pyrazolones.

The IL-1Ra fusion proteins described herein may be used in combination (pretreatment, post-treatment, or concurrent treatment) with any of one or more salicylic acid derivatives, prodrug esters or pharmaceutically acceptable salts thereof. Such salicylic acid derivatives, prodrug esters and pharmaceutically acceptable salts thereof comprise: acetaminosalol, aloxiprin, aspirin, benorylate, bromosaligenin, calcium acetylsalicylate, choline magnesium trisalicylate, magnesium salicylate, choline salicylate, diflusinal, etersalate, fendosal, gentisic acid, glycol salicylate, imidazole salicylate, lysine acetylsalicylate, mesalamine, morpholine salicylate, 1-naphthyl salicylate, olsalazine, parsalmide, phenyl acetylsalicylate, phenyl salicylate, salacetamide, salicylamide O-acetic acid, salsalate, sodium salicylate and sulfasalazine. Structurally related salicylic acid derivatives having similar analgesic and anti-inflammatory properties are also intended to be encompassed by this group.

In an additional specific embodiment, the present invention is directed to the use of an IL-1Ra fusion proteins in combination (pretreatment, post-treatment, or concurrent treatment) with any of one or more propionic acid derivatives, prodrug esters or pharmaceutically acceptable salts thereof. The propionic acid derivatives, prodrug esters, and pharmaceutically acceptable salts thereof comprise: alminoprofen, benoxaprofen, bucloxic acid, carprofen, dexindoprofen, fenoprofen, flunoxaprofen, fluprofen, flurbiprofen, furcloprofen, ibuprofen, ibuprofen aluminum, ibuproxam, indoprofen, isoprofen, ketoprofen, loxoprofen, miroprofen, naproxen, naproxen sodium, oxaprozin, piketoprofen, pimeprofen, pirprofen, pranoprofen, protizinic acid, pyridoxiprofen, suprofen, tiaprofenic acid and tioxaprofen. Structurally related propionic acid derivatives having similar analgesic and anti-inflammatory properties are also intended to be encompassed by this group.

In yet another specific embodiment, the present invention is directed to the use of an IL-1Ra fusion proteins in combination (pretreatment, post-treatment, or concurrent treatment) with any of one or more acetic acid derivatives, prodrug esters or pharmaceutically acceptable salts thereof. The acetic acid derivatives, prodrug esters, and pharmaceutically acceptable salts thereof comprise: acemetacin, alclofenac, amfenac, bufexamac, cinmetacin, clopirac, delmetacin, diclofenac potassium, diclofenac sodium, etodolac, felbinac, fenclofenac, fenclorac, fenclozic acid, fentiazac, furofenac, glucametacin, ibufenac, indomethacin, isofezolac, isoxepac, lonazolac, metiazinic acid, oxametacin, oxpinac, pimetacin, proglumetacin, sulindac, talmetacin, tiaramide, tiopinac, tolmetin, tolmetin sodium, zidometacin and zomepirac. Structurally related acetic acid derivatives having similar analgesic and anti-inflammatory properties are also intended to be encompassed by this group.

In another specific embodiment, the present invention is directed to the use of an IL-1Ra fusion proteins in combination (pretreatment, post-treatment, or concurrent treatment) with any of one or more fenamic acid derivatives, prodrug esters or pharmaceutically acceptable salts thereof. The fenamic acid derivatives, prodrug esters and pharmaceutically acceptable salts thereof comprise: enfenamic acid, etofenamate, flufenamic acid, isonixin, meclofenamic acid, meclofenamate sodium, medofenamic acid, mefenamic acid, niflumic acid, talniflumate, terofenamate, tolfenamic acid and ufenamate. Structurally related fenamic acid derivatives having similar analgesic and anti-inflammatory properties are also intended to be encompassed by this group.

In an additional specific embodiment, the present invention is directed to the use of an IL-1Ra fusion proteins in combination (pretreatment, post-treatment, or concurrent treatment) with any of one or more carboxylic acid derivatives, prodrug esters or pharmaceutically acceptable salts thereof. The carboxylic acid derivatives, prodrug esters, and pharmaceutically acceptable salts thereof which can be used comprise: clidanac, diflunisal, flufenisal, inoridine, ketorolac and tinoridine. Structurally related carboxylic acid derivatives having similar analgesic and anti-inflammatory properties are also intended to be encompassed by this group.

In yet another specific embodiment, the present invention is directed to the use of an IL-1Ra fusion proteins in combination (pretreatment, post-treatment, or concurrent treatment) with any of one or more butyric acid derivatives, prodrug esters or pharmaceutically acceptable salts thereof. The butyric acid derivatives, prodrug esters, and pharmaceutically acceptable salts thereof comprise: bumadizon, butibufen, fenbufen and xenbucin. Structurally related butyric acid derivatives having similar analgesic and anti-inflammatory properties are also intended to be encompassed by this group.

In another specific embodiment, the present invention is directed to the use of an IL-1Ra fusion proteins in combination (pretreatment, post-treatment, or concurrent treatment) with any of one or more oxicams, prodrug esters, or pharmaceutically acceptable salts thereof. The oxicams, prodrug esters, and pharmaceutically acceptable salts thereof comprise: droxicam, enolicam, isoxicam, piroxicam, sudoxicam, tenoxicam and 4-hydroxyl-1,2-benzothiazine 1,1-dioxide 4-(N-phenyl)-carboxamide. Structurally related oxicams having similar analgesic and anti-inflammatory properties are also intended to be encompassed by this group.

In still another specific embodiment, the present invention is directed to the use of an IL-1Ra fusion proteins in combination (pretreatment, post-treatment, or concurrent treatment) with any of one or more pyrazoles, prodrug esters, or pharmaceutically acceptable salts thereof. The pyrazoles, prodrug esters, and pharmaceutically acceptable salts thereof which may be used comprise: difenamizole and epirizole. Structurally related pyrazoles having similar analgesic and anti-inflammatory properties are also intended to be encompassed by this group.

In an additional specific embodiment, the present invention is directed to the use of an IL-1Ra fusion proteins in combination (pretreatment, post-treatment or, concurrent treatment) with any of one or more pyrazolones, prodrug esters, or pharmaceutically acceptable salts thereof. The pyrazolones, prodrug esters and pharmaceutically acceptable salts thereof which may be used comprise: apazone, azapropazone, benzpiperylon, feprazone, mofebutazone, morazone, oxyphenbutazone, phenylbutazone, pipebuzone, propylphenazone, ramifenazone, suxibuzone and thiazolinobutazone. Structurally related pyrazalones having similar analgesic and anti-inflammatory properties are also intended to be encompassed by this group.

In another specific embodiment, the present invention is directed to the use of an IL-1Ra fusion proteins in combination (pretreatment, post-treatment, or concurrent treatment) with any of one or more of the following NSAIDs: .epsilon.-acetamidocaproic acid, S-adenosyl-methionine, 3-amino-4-hydroxybutyric acid, amixetrine, anitrazafen, antrafenine, bendazac, bendazac lysinate, benzydamine, beprozin, broperamole, bucolome, bufezolac, ciproquazone, cloximate, dazidamine, deboxamet, detomidine, difenpiramide, difenpyramide, difisalamine, ditazol, emorfazone, fanetizole mesylate, fenflumizole, floctafenine, flumizole, flunixin, fluproquazone, fopirtoline, fosfosal, guaimesal, guaiazolene, isonixim, lefetamine HCl, leflunomide, lofemizole, lotifazole, lysin clonixinate, meseclazone, nabumetone, nictindole, nimesulide, orgotein, orpanoxin, oxaceprol, oxapadol, paranyline, perisoxal, perisoxal citrate, pifoxime, piproxen, pirazolac, pirfenidone, proquazone, proxazole, thielavin B, tiflamizole, timegadine, tolectin, tolpadol, tryptamid and those designated by company code number such as 480156S, AA861, AD1590, AFP802, AFP860, A177B, AP504, AU8001, BPPC, BW540C, CHINOIN 121, CN100, EB382, EL508, F1044, FIK-506, GV3658, ITF182, KCNTEI6090, KME4, LA2851, MR714, MR897, MY309, ONO3144, PR823, PV102, PV108, R830, RS2131, SCR152, SH440, SIR133, SPAS510, SQ27239, ST281, SY6001, TA60, TAI-901 (4-benzoyl-1-indancarboxylic acid), TVX2706, U60257, UR2301 and WY41770. Structurally related NSAIDs having similar analgesic and anti-inflammatory properties to the NSAIDs are also intended to be encompassed by this group.

In still another specific embodiment, the present invention is directed to the use of an IL-1Ra fusion proteins in combination (pretreatment, post-treatment or concurrent treatment) with any of one or more corticosteroids, prodrug esters or pharmaceutically acceptable salts thereof for the treatment of the diseases and disorders recited herein, including acute and chronic inflammation such as rheumatic diseases, graft versus host disease and multiple sclerosis. Corticosteroids, prodrug esters and pharmaceutically acceptable salts thereof include hydrocortisone and compounds which are derived from hydrocortisone, such as 21-acetoxypregnenolone, alclomerasone, algestone, amcinonide, beclomethasone, betamethasone, betamethasone valerate, budesonide, chloroprednisone, clobetasol, clobetasol propionate, clobetasone, clobetasone butyrate, clocortolone, cloprednol, corticosterone, cortisone, cortivazol, deflazacon, desonide, desoximerasone, dexamethasone, diflorasone, diflucortolone, difluprednate, enoxolone, fluazacort, flucloronide, flumethasone, flumethasone pivalate, flucinolone acetonide, flunisolide, fluocinonide, fluorocinolone acetonide, fluocortin butyl, fluocortolone, fluocortolone hexanoate, diflucortolone valerate, fluorometholone, fluperolone acetate, fluprednidene acetate, fluprednisolone, flurandenolide, formocortal, halcinonide, halometasone, halopredone acetate, hydro-cortamate, hydrocortisone, hydrocortisone acetate, hydro-cortisone butyrate, hydrocortisone phosphate, hydrocortisone 21-sodium succinate, hydrocortisone tebutate, mazipredone, medrysone, meprednisone, methylprednisolone, mometasone furoate, paramethasone, prednicarbate, prednisolone, prednisolone 21-diedryaminoacetate, prednisolone sodium phosphate, prednisolone sodium succinate, prednisolone sodium 21-m-sulfobenzoate, prednisolone sodium 21-stearoglycolate, prednisolone tebutate, prednisolone 21-trimethylacetate, prednisone, prednival, prednylidene, prednylidene 21-diethylaminoacetate, tixocortol, triamcinolone, triamcinolone acetonide, triamcinolone benetonide and triamcinolone hexacetonide. Structurally related corticosteroids having similar analgesic and anti-inflammatory properties are also intended to be encompassed by this group.

In another specific embodiment, the present invention is directed to the use of an IL-1Ra fusion proteins in combination (pretreatment, post-treatment, or concurrent treatment) with any of one or more slow-acting antirheumatic drugs (SAARDs) or disease modifying antirheumatic drugs (DMARDS), prodrug esters, or pharmaceutically acceptable salts thereof for the treatment of the diseases and disorders recited herein, including acute and chronic inflammation such as rheumatic diseases, graft versus host disease and multiple sclerosis. SAARDs or DMARDS, prodrug esters and pharmaceutically acceptable salts thereof comprise: allocupreide sodium, auranofin, aurothioglucose, aurothioglycanide, azathioprine, brequinar sodium, bucillamine, calcium 3-aurothio-2-propanol-1-sulfonate, chlorambucil, chloroquine, clobuzarit, cuproxoline, cyclo-phosphamide, cyclosporin, dapsone, 15-deoxyspergualin, diacerein, glucosamine, gold salts (e.g., cycloquine gold salt, gold sodium thiomalate, gold sodium thiosulfate), hydroxychloroquine, hydroxychloroquine sulfate, hydroxyurea, kebuzone, levamisole, lobenzarit, melittin, 6-mercaptopurine, methotrexate, mizoribine, mycophenolate mofetil, myoral, nitrogen mustard, D-penicillamine, pyridinol imidazoles such as SKNF86002 and SB203580, rapamycin, thiols, thymopoietin and vincristine. Structurally related SAARDs or DMARDs having similar analgesic and anti-inflammatory properties are also intended to be encompassed by this group.

In another specific embodiment, the present invention is directed to the use of an IL-1Ra fusion proteins in combination (pretreatment, post-treatment, or concurrent treatment) with any of one or more COX2 inhibitors, prodrug esters or pharmaceutically acceptable salts thereof for the treatment of the diseases and disorders recited herein, including acute and chronic inflammation. Examples of COX2 inhibitors, prodrug esters or pharmaceutically acceptable salts thereof include, for example, celecoxib. Structurally related COX2 inhibitors having similar analgesic and anti-inflammatory properties are also intended to be encompassed by this group. Examples of COX-2 selective inhibitors include but not limited to etoricoxib, valdecoxib, celecoxib, licofelone, lumiracoxib, rofecoxib, and the like.

In still another specific embodiment, the present invention is directed to the use of an IL-1Ra fusion proteins in combination (pretreatment, post-treatment, or concurrent treatment) with any of one or more antimicrobials, prodrug esters or pharmaceutically acceptable salts thereof for the treatment of the diseases and disorders recited herein, including acute and chronic inflammation. Antimicrobials include, for example, the broad classes of penicillins, cephalosporins and other beta-lactams, aminoglycosides, azoles, quinolones, macrolides, rifamycins, tetracyclines, sulfonamides, lincosamides and polymyxins. The penicillins include, but are not limited to penicillin G, penicillin V, methicillin, nafcillin, oxacillin, cloxacillin, dicloxacillin, floxacillin, ampicillin, ampicillin/sulbactam, amoxicillin, amoxicillin/clavulanate, hetacillin, cyclacillin, bacampicillin, carbenicillin, carbenicillin indanyl, ticarcillin, ticarcillin/clavulanate, azlocillin, meziocillin, peperacillin, and mecillinam. The cephalosporins and other beta-lactams include, but are not limited to cephalothin, cephapirin, cephalexin, cephradine, cefazolin, cefadroxil, cefaclor, cefamandole, cefotetan, cefoxitin, ceruroxime, cefonicid, ceforadine, cefixime, cefotaxime, moxalactam, ceftizoxime, cetriaxone, cephoperazone, ceftazidime, imipenem and aztreonam. The aminoglycosides include, but are not limited to streptomycin, gentamicin, tobramycin, amikacin, netilmicin, kanamycin and neomycin. The azoles include, but are not limited to fluconazole. The quinolones include, but are not limited to nalidixic acid, norfloxacin, enoxacin, ciprofloxacin, ofloxacin, sparfloxacin and temafloxacin. The macrolides include, but are not limited to erythomycin, spiramycin and azithromycin. The rifamycins include, but are not limited to rifampin. The tetracyclines include, but are not limited to spicycline, chlortetracycline, clomocycline, demeclocycline, deoxycycline, guamecycline, lymecycline, meclocycline, methacycline, minocycline, oxytetracycline, penimepicycline, pipacycline, rolitetracycline, sancycline, senociclin and tetracycline. The sulfonamides include, but are not limited to sulfanilamide, sulfamethoxazole, sulfacetamide, sulfadiazine, sulfisoxazole and co-trimoxazole (trimethoprim/sulfamethoxazole). The lincosamides include, but are not limited to clindamycin and lincomycin. The polymyxins (polypeptides) include, but are not limited to polymyxin B and colistin.

It should be noted that the section headings are used herein for organizational purposes only, and are not to be construed as in any way limiting the subject matter described. All references cited herein are incorporated by reference in their entirety for all purposes.

The Examples that follow are merely illustrative of certain embodiments of the invention, and are not to be taken as limiting the invention, which is defined by the appended claims.

### EXAMPLES

### Example 1

### Format, Production and Purification of Trimeric IL-1Ra

It has been previously been shown that IL-1Ra can be produced as recombinant protein in E. coli. (Steinkasserer et al 1992. FEBS 310:63-65). The protein is very stable and refolds efficiently. Isoforms of IL-1Ra with additional amino acids in the N-terminal have been also described (Haskill et al 1991, PNAS 88:3681-3685; Muzio et al 1995, JEM 182, 623-628)). These molecules bind IL-1R as well as the mature secreted form indicating that it is possible to fuse extra peptide to the N-terminal of the antagonist without compromising the binding to the receptor. Crystal structure analysis of IL-1Ra interaction with IL-1R also supports that N-terminal alterations do not affect interactions with IL1R (Schreuder et al 1997, Nature 386: 190-194). IL-1Ra was cloned from a human cDNA library derived from bone marrow and/or human placenta.

Trimeric IL-1Ra was designed as a C-terminal fusion to the Trip-trimerization unit. Eight different fusion proteins were designed, four with full length trimerization units (Trip) and four with a nine amino acid truncation of the trimerization unit (I10Trip). IL-1ra was than fused with either trimerization unit using four different C-terminal fusions. C-terminal variations termed Trip V, Trip T, Trip Q and Trip K allow for unique presentation of the CTLD domains on the trimerization domain. The Trip K variant is the longest construct and contains the longest and most flexible linker between the CTLD and the trimerization domain. Trip V, Trip T, Trip Q represent fusions of the CTLD molecule directly onto the trimerization module without any structural flexibility but are turning the CTLD molecule ⅓^{rd} going from Trip V to Trip T and from Trip T to Trip Q. This is due to the fact that each of these amino acids is in an α-helical turn and 3.2 aa are needed for a full turn

The following proteins were produced as the following Granzyme B cleavable fusion proteins in BL21 AI bacteria. The underlined portions denotes the trimerization unit, and the bold part denotes the IL-1Ra part:

CII-H6-GrB-GG-TripK-IL-1ra:

(SEQ ID NO: 47)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQTVSLK
RPSGRKSSKMQAFRIWDVNQKTFYLRNNQLVAGYLQGPNVNLEEKIDVVP
IEPHALFLGIHGGKMCLSCVKSGDETRLQLEAVNITDLSENRKQDKRFAF
IRSDSGPTTSFESAACPGWFLCTAMEADQPVSLTNMPDEGVMVTKFYFQE
DE;
CII-H6-GrB-GG-TripV-IL-1ra:

(SEQ ID NO: 48)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQTVRPS
GRKSSKMQAFRIWDVNQKTFYLRNNQLVAGYLQGPNVNLEEKIDVVPIEP
HALFLGIHGGKMCLSCVKSGDETRLQLEAVNITDLSENRKQDKRFAFIRS
DSGPTTSFESAACPGWFLCTAMEADQPVSLTNMPDEGVMVTKFYFQEDE;
CII-H6-GrB-GG-TripT-IL-1ra:

(SEQ ID NO: 49)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQTRPSG
RKSSKMQAFRIWDVNQKTFYLRNNQLVAGYLQGPNVNLEEKIDVVPIEPH
ALFLGIHGGKMCLSCVKSGDETRLQLEAVNITDLSENRKQDKRFAFIRSD
SGPTTSFESAACPGWFLCTAMEADQPVSLTNMPDEGVMVTKFYFQEDE;
CII-H6-GrB-GG-TripQ-IL-1ra:

(SEQ ID NO: 50)

MVRANKRNEALRIESALLNKIAMLGTEKTAEGGSHHHHHHGSIEPDGGEG
PTQKPKKIVNAKKDVVNTKMFEELKSRLDTLAQEVALLKEQQALQRPSGR
KSSKMQAFRIWDVNQKTFYLRNNQLVAGYLQGPNVNLEEKIDVVPIEPHA
LFLGIHGGKMCLSCVKSGDETRLQLEAVNITDLSENRKQDKRFAFIRSDS
GPTTSFESAACPGWFLCTAMEADQPVSLTNMPDEGVMVTKFYFQEDE;
CII-H6-GrB-I10-TripK-IL-1ra:

(SEQ ID NO: 51)

MVRANKRNEALRIESALLNKIAMLGTEKTAEGGSHHHHHHGSIEPDIVNA
KKDVVNTKMFEELKSRLDTLAQEVALLKEQQALQTVSLKRPSGRKSSKMQ
AFRIWDVNQKTFYLRNNQLVAGYLQGPNVNLEEKIDVVPIEPHALFLGIH
GGKMCLSCVKSGDETRLQLEAVNITDLSENRKQDKRFAFIRSDSGPTTSF
ESAACPGWFLCTAMEADQPVSLTNMPDEGVMVTKFYFQEDE;
CII-H6-GrB-I10-TripV-IL-1ra:

(SEQ ID NO: 52)

MVRANKRNEALRIESALLNKIAMLGTEKTAEGGSHHHHHHGSIEPDIVNA
KKDVVNTKMFEELKSRLDTLAQEVALLKEQQALQTVRPSGRKSSKMQAFR
IWDVNQKTFYLRNNQLVAGYLQGPNVNLEEKIDVVPIEPHALFLGIHGGK
MCLSCVKSGDETRLQLEAVNITDLSENRKQDKRFAFIRSDSGPTTSFESA
ACPGWFLCTAMEADQPVSLTNMPDEGVMVTKFYFQEDE;
CII-H6-GrB-I10-TripT-IL-1ra:

(SEQ ID NO: 53)

MVRANKRNEALRIESALLNKIAMLGTEKTAEGGSHHHHHHGSIEPDIVNA
KKDVVNTKMFEELKSRLDTLAQEVALLKEQQALQTRPSGRKSSKMQAFRI
WDVNQKTFYLRNNQLVAGYLQGPNVNLEEKIDVVPIEPHALFLGIHGGKM
CLSCVKSGDETRLQLEAVNITDLSENRKQDKRFAFIRSDSGPTTSFESAA
CPGWFLCTAMEADQPVSLTNMPDEGVMVTKFYFQEDE;
and
CII-H6-GrB-I10-TripQ-IL-1ra:

(SEQ ID NO: 54)

MVRANKRNEALRIESALLNKIAMLGTEKTAEGGSHHHHHHGSIEPDIVNA
KKDVVNTKMFEELKSRLDTLAQEVALLKEQQALQRPSGRKSSKMQAFRIW
DVNQKTFYLRNNQLVAGYLQGPNVNLEEKIDVVPIEPHALFLGIHGGKMC
LSCVKSGDETRLQLEAVNITDLSENRKQDKRFAFIRSDSGPTTSFESAAC
PGWFLCTAMEADQPVSLTNMPDEGVMVTKFYFQEDE

All constructs were captured on NiNTA Superflow (Qiagen), refolded and further purified on SP-Sepharose FF (GE Heathcare). From expression in shake flask or from a fermentation of the trimeric IL1-Ra, inclusion bodies were purified. Packed cell pellet was homogenized in lysis-buffer (50 mM Tris-HCl, pH 8.0, 25 w/v % Sucrose, 1 mM EDTA) by sonication (50 g wet cell pellet per 100 mL lysis buffer). Then 100 mg lysozyme per 100 mL lysis-buffer was added and mixed before the sample was left for 15 min at R.T. The sample was then sonicated for 2-5 min with mixing in between. Detergent buffer (0.2 M NaCl, 1 w/v % Deoxycholate, Na salt, 1 w/v % Nonidet P40, 20 mM Tris-HCl, pH 7.5, 2 mM EDTA) was added and the sample is mixed and sonified again. The inclusion bodies were recovered by centrifugation for 25 min at 8.000 rpm, 4° C. The supernatant was stored at 4° C. and the pellet resuspended in 100 mL TRITON®X-100 buffer (0.5 w/v % TRITON® X-100, 1 mM EDTA, pH 8) per 50 g original cell pellet. Inclusion bodies were recovered by centrifugation for 25 min at 8.000 rpm, 4° C. and the supernatant was stored at 4° C. The TRITON® X-100 buffer wash is repeated once more and the inclusion bodies were recovered by centrifugation for 5 min at 12.000 rpm, 4° C.

The inclusion bodies were re-suspended in 30 mL denaturing buffer/gram original cell paste (6 M urea, 10 mM EDTA, 20 mM Tris/HCl and 20 mM β-Mercaptoethanol, pH 8.0) at 28° C. for 2 h. The suspension was centrifuged at 7500 g for 15 min to remove insoluble material. Following this CaCl₂ was added to 20 mM final concentration and the solution was applied to a 100 mL Ni-NTA Superflow column equillibrated in NTA buffer (8 M Urea; 1000 mM NaCl; 50 mM Tris HCl pH 8.0; 5 mM β-Mercaptoethanol) and washed until a stable baseline was obtained. A further wash with 250 mL guanidine-HCl, 50 mM Tris-HCl pH 8.0, 5 mM β-Mercaptoethanol followed by wash with 100 mL buffer NTA.

Two refolding methods have been used, dialysis refolding and on-column refolding and both have yielded pure and soluble protein. For dialysis refolding the resuspended inclusion bodies was used directly for dialysis over into 1×PBS containing 3 M urea, 1 mM EDTA, pH 7.2 over night. The day after the dialysis was continued into 1×PBS containing 0 M urea, 1 mM EDTA, pH 7.2.

For on-column refolding the washed Ni-NTA Superflow column with protein bound, the resin was washed with 4 CV ml 1×PBS containing 3 M urea, pH 7.2 before a linear gradient of 10 CV 1×PBS containing 3 M urea, pH 7.2 and 10 CV 1×PBS containing 0 M urea, pH 7.2 was run. To recover the refolded trimeric IL-1ra, the column eluted with 1×PBS, 10 mM EDTA, pH 6.0 and fraction were collected.

Following refolding cleavage with recombinant human Granzyme B was performed by adjusting the pH in the eluate to 7.5 with NaOH before Granzyme B was added at a 1:500 ratio (granzyme/protein) and incubated at 25° C. over night. The progress was followed by SDS-PAGE.

Finally, the cleaved protein was purified using SP-Sepharose FF (GE Healthcare) cation exchange step. A 50 mL SP-Sepharose FF was packed and equilibrated in buffer A (1×PBS, 1 mM EDTA pH 5,5) until stabile basis line was obtained. The cleavage reaction was diluted 1:3 with buffer A and loaded on the column followed by a wash in buffer A until stabile basis line was monitored. A gradient from 10 CV buffer A to 10 CV Buffer B (1×PBS, 1 mM EDTA+0.5 M NaCl pH 5.5) was setup and fractions collected in 5 mL. Protein containing fractions were analysed on SDS-PAGE before pooling the protein product.

Alternatively, the supernatant from the above inclusion body preparation were used to purify the protein. The soluble Trimeric IL1-ra in the supernatant was purified on Ni-NTA Superflow (Qiagen) column equilibrated in Buffer A (20 mM Tris HCL, 50 mM NaCl pH 8.0. A pool was made of the washes from the inclusion body purification and it was centrifuged at 10000 rpm for 10 min before CaCl₂ was added to 5 mM and Tris-HCl to 20 mM and the pH adjusted to 6.0 with HClNaOH. The pool was loaded on the column and washed in buffer A until stabile basis line. Following a wash in buffer A+1 M NaCl until stabile basis line, the bound protein was eluted with buffer A+20 mM EDTA and fractions were collected. Hereafter the protein pool was cleaved with Granzyme B and polished on a SP-Sepharose FF column as described above. The soluble fraction of CII-H6-GrB-GG-TripK-IL-1ra from 3L expression culture gave a final yield of 95 mg of TripK-IL-1Ra following (˜250 mg CII-H6-GrB-TripK-IL-1Ra after capture Ni-NTA Superflow (Qiagen). Since the yield and purity of the protein from the soluble fraction was significantly better than doing refolding, this path was chosen following the initial construct testing.

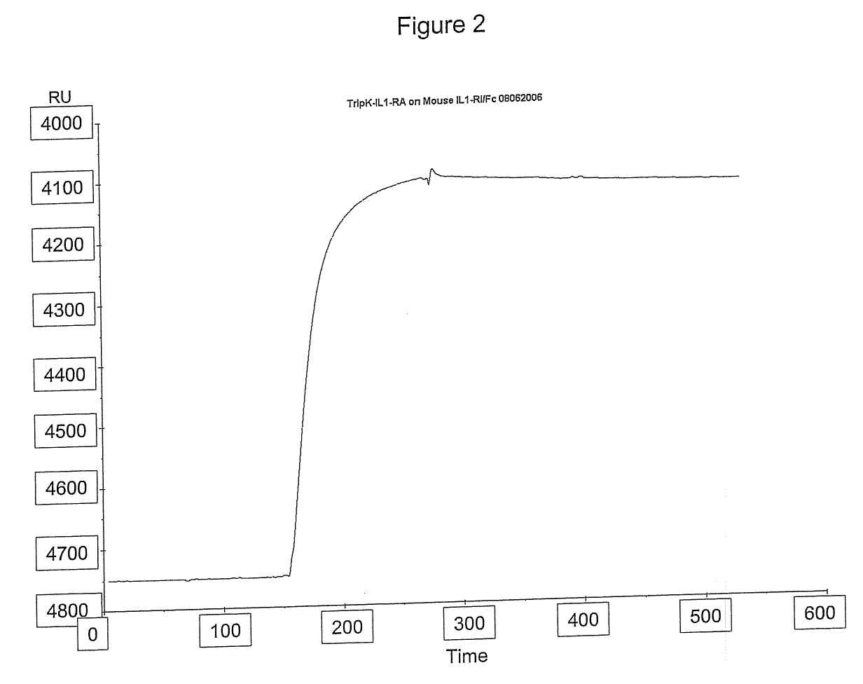

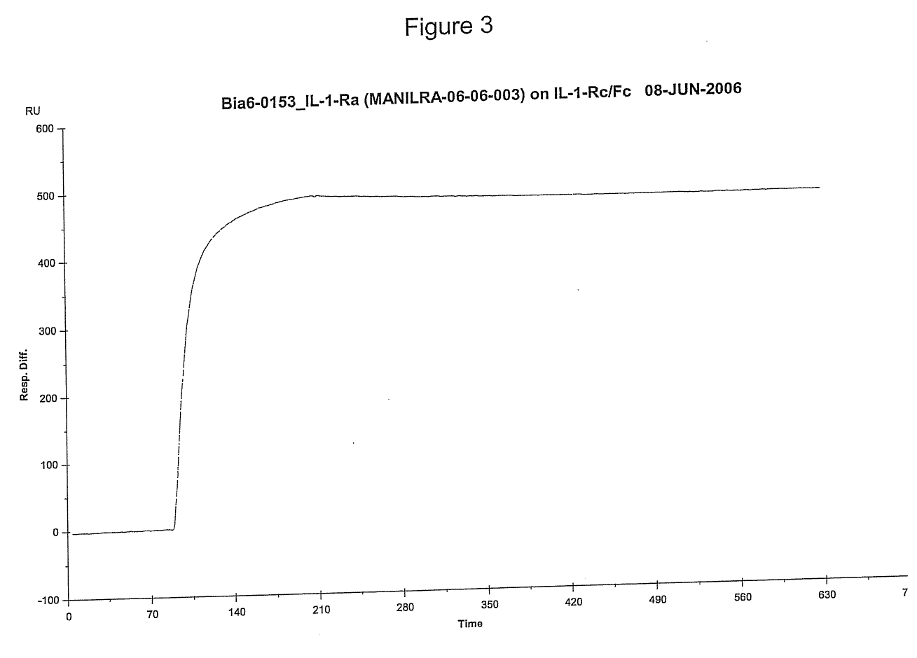

The ability of the refolded protein to bind to IL-1 Receptor 1 was analysed on a Biacore 3000 (Biacore, Uppsala, Sweden) where mouse IL1-RI/Fc was coupled to CM5 sensor chips and binding of soluble TripK-IL-1ra to IL-1RI protein was measured. Results of uncleaved CII-H6-GrB-TripK-IL-1ra refolding by dialysis are shown in FIG. 2 and uncleaved CII-H6-GrB-TripK-IL-1ra on-column NiNTA refolding is shown in FIG. 3. The cleavage and purification assays produced the trimeric IL-1Ra compounds of SEQ ID NOs: 47-54.

### Example 2

### Trimeric IL-1Ra Compounds Ability to Inhibit IL-1 Induction of IL-8 in U937 Cells

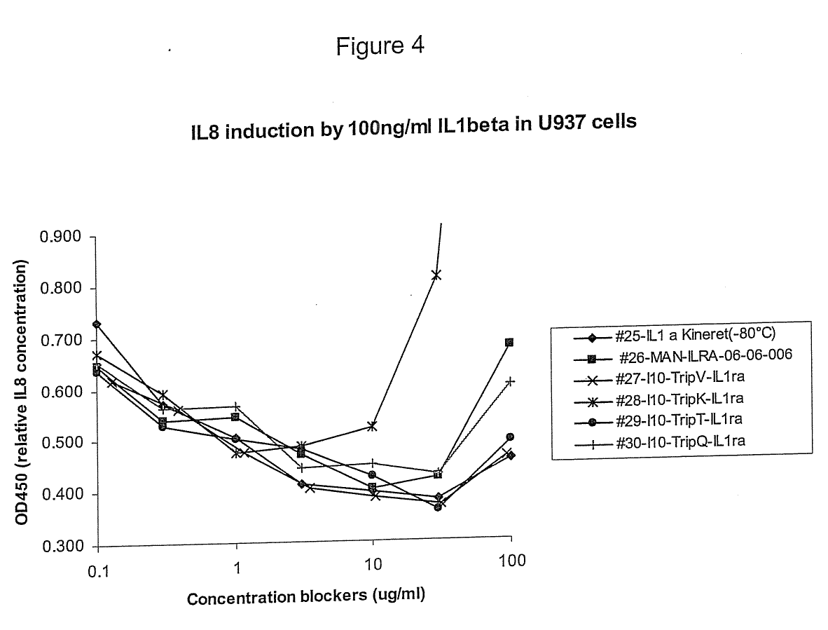

GG-TripV-IL-1ra (trip V-IL1Ra), GG-TripK-IL-1ra (trip K-IL1Ra), GG-TripT-IL-1ra (trip T-IL1Ra) and CII-H6-GrB-GG-TripT-IL-1ra (trip Q-IL1Ra) were further analysed for their ability to inhibit IL-1 induction of IL-8 in U937 cells. Results are shown in FIG. 4.

The compounds are essentially equally effective in blocking the response and they appear all to be as effective as KINERET® (when compared on w/w). Due to buffer effects in the assay, at the highest protein concentration used (100 μg/mL) IL-8 production increases instead of further decreasing. Based on several in vitro efficacy assays as well as Biacore assays, it was determined that TripT IL1Ra was the best compound based on blocking and binding efficacy as well as production yields.

### Example 3

### Pegylated Trimeric IL-1Ra Compounds

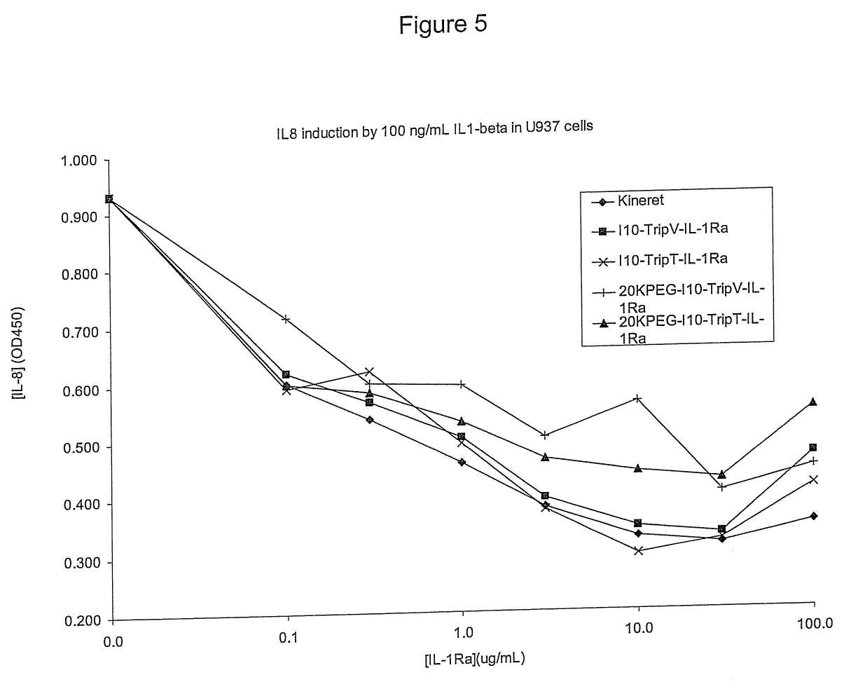

Since the in vivo half life is a crucial parameter in the efficacy of KINERET® (KINERET® has only a half life in humans of 4-6 hours and has therefore, to be applied once daily) the ability to pegylate the TripT IL1Ra by N-terminal pegylation was tested. The trimeric IL1-Ra is pegylated at the N terminus. Trimeric IL1-Ra antagonist proteins after the final step of the purification procedure described above were used as starting point for pegylations. The proteins were buffer changed into PBS buffer pH 6.0 for the pegylation reaction. The protein concentration in the reaction was between 0.5 and 3.5 mg/mL and a 5-10 molar excess of mPeg5K-Aldehyde or mPeg20K-Aldehyde (Nektar) supplemented with 20 mM cyanoborohydride (NaCNBH3) was used. The reaction was carried out at 20° C. for 16 hours. Following the reaction mixture was applied to Source 15S column (GE Healtcare) to purify the monopegylated form. As shown in FIG. 5, antagonistic activity of the pegylated version was reduced compared to the unpegylated protein. However, the pegylated protein still has good IL1 blocking efficacy.

### Example 4

### Pharmacokinetic Analysis of Trimeric IL1Ra Proteins in Male Lewis Rats After i.v. Infection

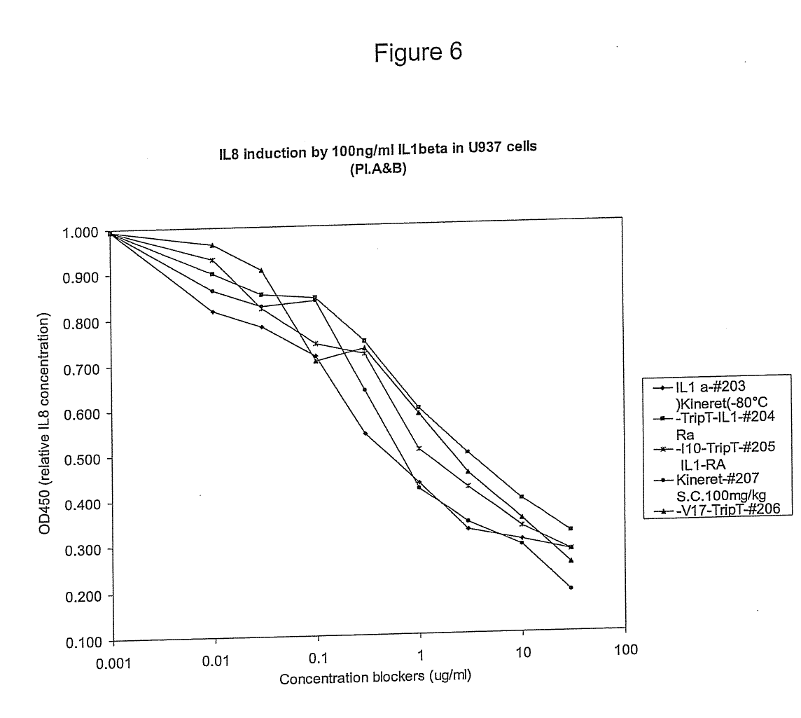

Three of the trimeric IL1Ra polypeptides described in the previous examples were chosen for pharmacokinetic analysis. The differences in the constructs were in the N-terminus of the trimerization domain: full length (FL), first nine amino acids truncated (I10) and the first 16 amino acids truncated (V17). The 10 construct represents a naturally occurring deletion variant of the trimerization domain and lacks the O-glycosylation site at Thr 4. The V17 derivative represents a deletion of the first exon encoding the trimerization domain and lacks a characterized heparin binding site. This site is also partially removed in the I10 construct. In vitro efficacy of the IL-1Ra molecules was verified in a U937 cell assay as shown in FIG. 6.

The pharmacokinetic profile of these three constructs polypeptides were analysed in Lewis rats after intravenous (i.v.) injections. The profiles obtained were compared to the pharmacokinetic profile of KINERET® in the same experiment. The pharmacokinetic study was conducted using four male Lewis rats per group, and the constructs that were used were FL IL-1Ra, I10 IL-1Ra, V17 IL-1Ra and KINERET®. Single i.v. doses of 100 mg/kg were given to the animals. The test compound was dissolved in vehicle (4.4 mM NaCltrate, pH 6.5, 93.8 mM NaCl, 0.33 mM EDTA, 0.7 g TWEEN®-80) and administered through the tail vein (vena sacralis media) or the hind paw vein (vena saphena).

Blood was then collected from four animals per time-point at baseline (zero hours) and 0.5, 1, 2, 4, 8, 12, 24, 48, 72 h post dosing. Blood samples of approximately 100 μl were collected from the tip of the tails in Microtainers™. Plasma was collected and transferred into polypropylene tubes. Plasma samples were then stored at <−70° C. until measurements were performed. Animals were then sacrificed by CO₂ inhalation and the carcasses were discarded without pathological examination. The IL-1Ra compound levels and KINERET® levels in plasma were then determined by ELISA.

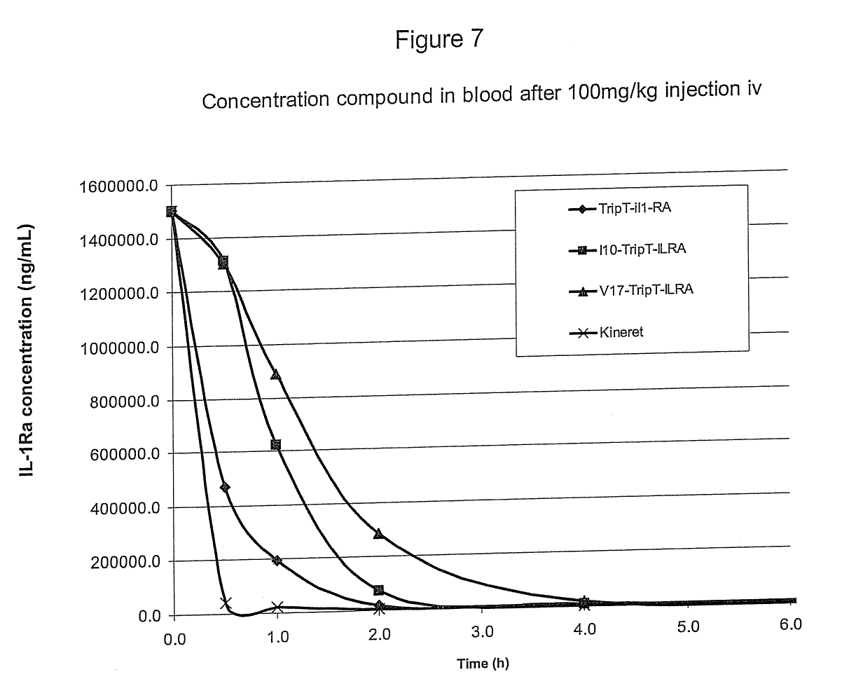

The average body weight of each rat was 250 grams. Assuming that the rat average blood volume was 16.5 mL a theoretical maximum initial concentration of the compounds of 1,500,000 ng/mL was calculated after i.v. injection. These concentrations are shown in FIG. 7. This starting level was used as starting value for the analysis. No observations of side effects or changes in animal well being were observed.

Following blood sampling at the above indicated time points, an ELISA assay was used to measure the injected protein in the blood samples. Based on these ELISA results, area under the curve (AUC) was used as a measure of drug exposure and the plasma half life were calculated using standard software. The areas under the curve are shown in Table 2 and the plasma half lives of the proteins are shown in Table 3.

TABLE 2

| | | AUC protein/AUC |
|---|---|---|
| Protein | AUC (ng/mL*h) | KINERET ® |
| FL IL1Ra | 809292 | 1.89 |
| I10 IL1Ra | 1637866 | 3.82 |
| V17 IL1Ra | 2177781 | 5.08 |
| KINERET ® | 428414 | 1 |

TABLE 3

| | | Half life protein/ |
|---|---|---|
| Protein | Half life (min.) | Half life KINERET ® |
| FL IL1Ra | 20 | 17 |
| I10 IL1Ra | 54 | 45 |
| V17 IL1Ra | 69 | 58 |
| KINERET ® | 1.2 | 1 |

These i.v. data indicate that the trimeric compounds have superior plasma half lives in comparison to KINERET®. The half life of KINERET® is about 1.2 minutes, whereas the half life of the V17 IL1Ra trimeric protein after i.v. injection is about 69 minutes. Dependent on the criteria used in the analysis the relative increase in AUC is between two-fold for FL IL1Ra trimer and five-fold for V17 IL1Ra trimer, indicating substantially improved drug exposure using the trimerized variants compared to KINERET®.

### Example 5

### Production of Met-I10-TripT-IL1ra and GG-V17-TripT-IL1ra and Rat CIA Model

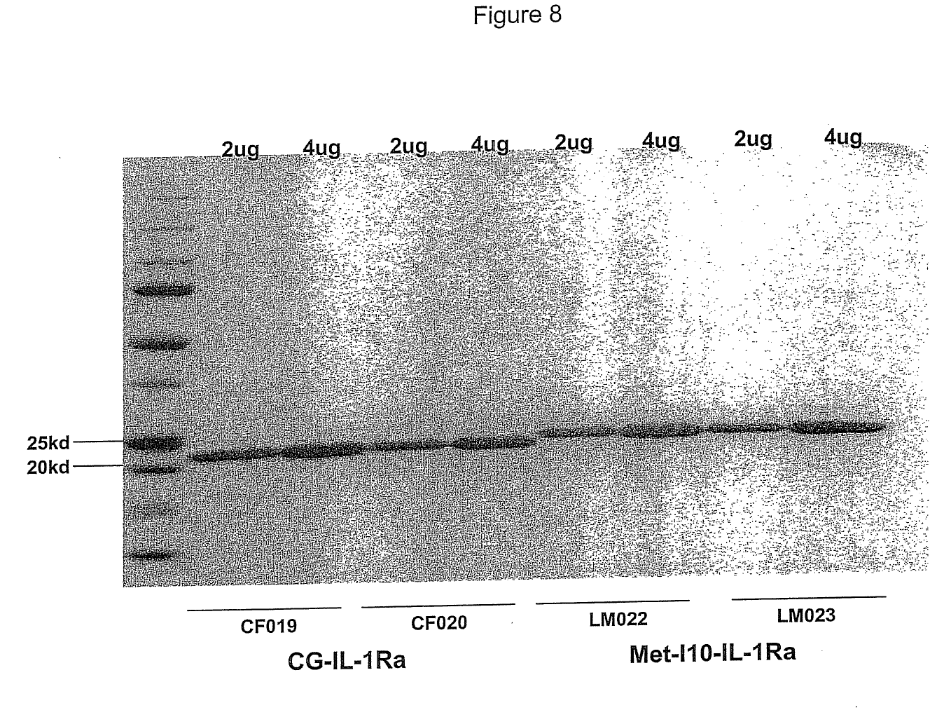

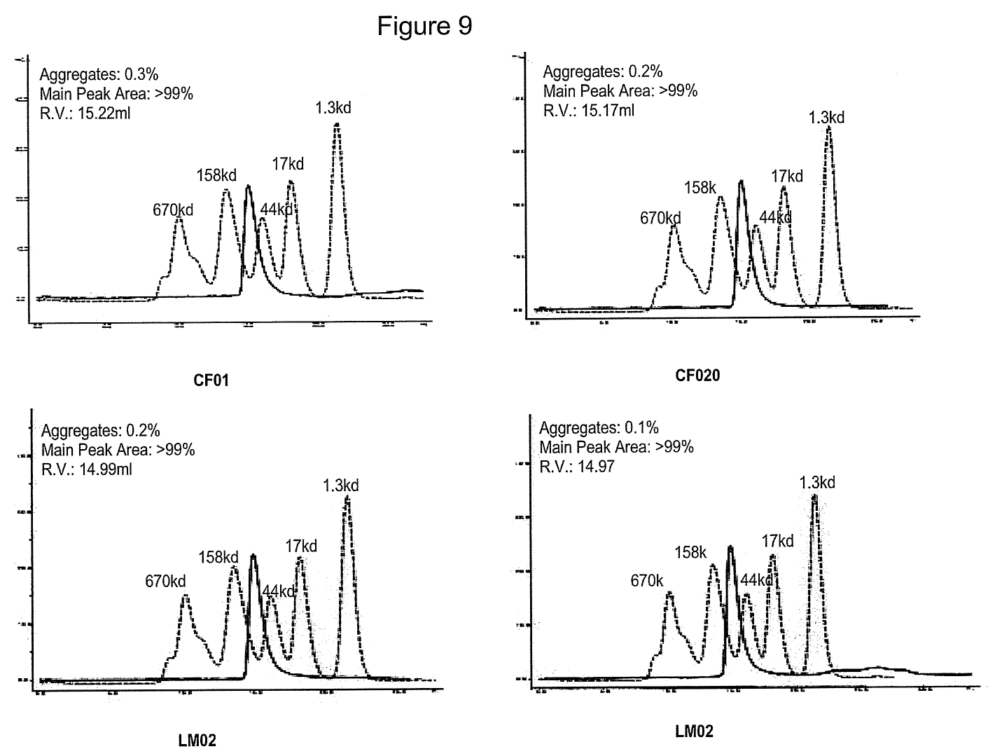

Both molecules were produced by BL21 AI bacteria in 10 L fermentor runs using either 2×TY medium (Met-I10-TripT-IL-1ra) or chemically defined minimal medium (GG-V17-TripT-IL-1ra). Cell pellets were obtained by centrifugation at 5887×g for 20 min, then resuspended in 10 mM Na₂HPO₄ pH 6. For Met-10-TripT-IL-1ra, the soluble cell fraction containing the protein of interest was obtained by high pressure homogenization (2×17.000 psi) followed by 10 min centrifugation at 10.000×g. The supernatant was diluted with 10 mM Na₂HPO₄ pH 7.4 and run over a SP-Sepharose FF column (cation exchange, GE Healthcare) followed by Q-Sepharose FF (anion exchange. GE Healthcare) using an AKTA fPLC. In a last step, proteins were run through a Mustang E filter (Pall) to remove endotoxin, followed by buffer exchange into PBS pH 7.4 and concentration to 50 mg/mL. The GG-V17-TripT-IL-1Ra protein was expressed as a fusion protein comprising an N-terminal booster domain, phage CII protein, followed by a human Granzyme B cleavage site. The GG-V17-TripT-IL-1Ra was purified from fermentation cell pellets by homogenization in lysis buffer containing lysozyme followed by centrifugation for 25 min at 8000 rpm. The supernatant was then run through a Fractogel® EMD Chelate (M) column (EMD Chemicals Inc.), and the eluate was buffer exchanged into 20 mM Tris pH 7.5, 150 mM NaCl. The protein fraction was then digested with recombinant human Granzyme B (made in house, ref to patent). After dilution with PBS pH 6, the proteins were purified using SP Sepharose FF followed by Mustang E filtration and Fractogel® EMD Chelate (M) column in flow through mode to remove the fusion tag and human Granzyme B. Final, the protein was buffer exchanged into PBS pH 7.4 and concentrated to 50 mg/mL. Yields for both Met-I10-TripT-IL-1ra and GG-V17-TripT-IL-1ra proteins were 3-5 g/L, purity >95% as determined by SDS-PAGE (FIG. 8), RP-HPLC and MS. Endotoxin levels were <3EU/mg as determined using a LAL assay (Lonza). Aggregates were <0.5% as determined by analytical SEC (FIG. 9) and host cell protein <6 ng/mL. Two batches (LM022, LM023) of Met-I10-TripT-IL-1ra and two batches (CF019, CF020) of GG-V17-TripT-IL-1ra were tested in above assays.

Female Lewis rats with 4-day established type II collagen arthritis were treated subcutaneously (SC), daily (QD) on arthritis days 1-3 with Vehicle (10 mM phosphate buffer pH 7.4), or equimolar amounts of IL-1ra administering either monomeric IL-1ra (100 mg/kg KINERET®), or trimerized IL1ra (120 mg/kg Met-I10-TripT-IL1ra, or 120 mg/kg GG-V17-TripT-IL1ra). In order to have only one set of controls, all rats in the QD groups were dosed with the respective vehicle (10 mM phosphate buffer pH 7.4, or sodium citrate buffer pH 6.5 for KINERET®) at the 2nd and 3rd dosings to keep manipulations constant. Animals were terminated on arthritis day 4. Efficacy evaluation was based on ankle caliper measurements, expressed as area under the curve (AUC), terminal hind paw weights and body weights (Bendele et al 2000, Arthritis+Rheumatism 43:2648-2659). All animals survived to study termination. Rats injected with KINERET® or its vehicle (CSEP) vocalized during the injection process thus suggesting that subcutaneous irritation was occurring. No vocalization occurred with any other injections.

Animals (8/group for arthritis, 4/group for normal), housed 4/cage, were anesthetized with Isoflurane and received subcutaneous/intradermal (SC/ID) injections with 300 μl of Freund's Incomplete Adjuvant (Difco, Detroit, Mich.) containing 2 mg/ml bovine type II collagen (Elastin Products, Owensville, Mo.) at the base of the tail and 2 sites on the back on days 0 and 6. Dosing by subcutaneous route (QD at 24 hour intervals) was initiated on arthritis day 1 and continued through day 3. Experimental groups were as shown in Table 4

TABLE 4

| | | QD SC Treatment 2.3 ml/kg, days 1-3, Dose volumes are |
|---|---|---|
| Group | N | based on equivalent IL-1ra molecules |
| 1 | 4 | Normal controls, vehicle (10 mM phosphate buffer pH |
| | | 7.4) TID |
| 2 | 8 | Arthritis + KINERET ® QD (100 mg/kg), vehicle (sodium |
| | | citrate buffer pH 6.5) at other times |
| 3 | 8 | Arthritis + Met-I10-TripT-IL1ra QD (120 mg/kg), vehicle |
| | | (10 mM phosphate buffer pH 7.4) at other times |
| 4 | 8 | Arthritis + V17-TripT-IL1ra QD (120 mg/kg), vehicle |
| | | (10 mM phosphate buffer pH 7.4) at other times |

Rats were weighed on days 0-4 of arthritis, and caliper measurements of ankles were taken every day beginning on day 0 of arthritis (study day 9). After final body weight measurement, animals were euthanized, and hind paws were transected at the level of the medial and lateral malleolus and weighed (paired).

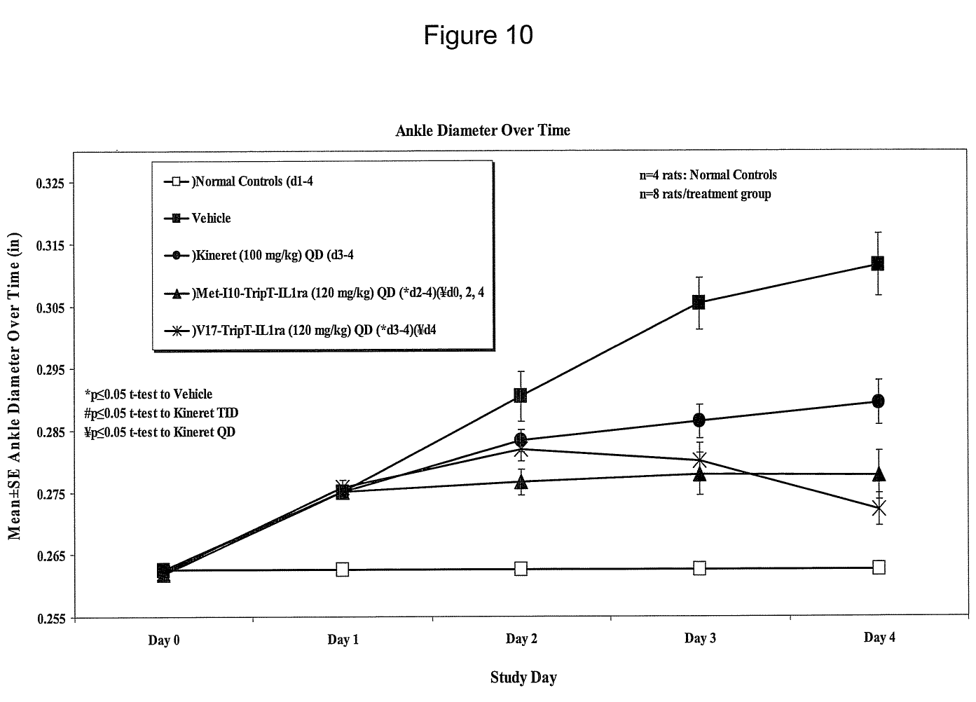

Significant reduction of ankle diameter was seen in rats treated with 100 mg/kg KINERET® QD (d3-4), 120 mg/kg Met-I10-TripT-IL1ra QD (d2-4), or 120 mg/kg GG-V17-TripT-IL1ra QD (d3-4), as compared to vehicle treated disease control animals. Reduction of ankle diameter AUC was significant for rats treated with 100 mg/kg KINERET® QD (34%), 120 mg/kg Met-I10-TripT-IL1ra QD (54%), or 120 mg/kg GG-V17-TripT-IL1ra QD (49%), as compared to vehicle treated disease control animals. Met-I10-TripT-IL1ra QD treatment resulted in significantly reduced ankle diameter AUC compared to KINERET® QD treatment (p<0.035 at the end of the study). Also, GG-V17-TripT-IL1ra QD treatment resulted in significantly reduced ankle diameter AUC compared to KINERET® QD treatment at the end of the study (p<0.001). (FIG. 10)

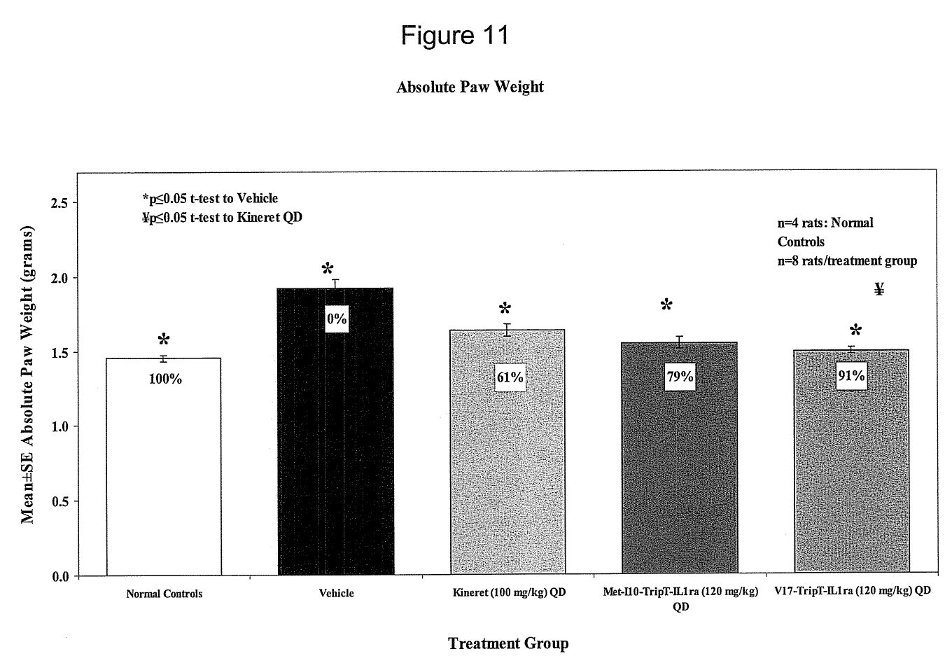

Reduction of final paw weight was significant for rats treated with 100 mg/kg KINERET® QD (61%), 120 mg/kg Met-I10-TripT-IL1ra QD (79%), or 120 mg/kg GG-V17-TripT-IL1ra QD (91%), as compared to vehicle treated disease control animals. GG-V17-TripT-IL1ra QD treatment resulted in significantly reduced final paw weights compared to KINERET® QD treatment (p<0.006). (FIG. 11)

Change in body weight was significantly increased toward normal for rats treated with 100 mg/kg KINERET® QD (54%), 120 mg/kg Met-I10-TripT-IL1ra QD (49%), or 120 mg/kg GG-V17-TripT-IL1ra QD (65%), as compared to vehicle treated disease control animals.

### Example 6

### Streptozocin (STZ)-Induced Diabetes Model

STZ (Sigma Aldrich) was administered once daily for five successive days at 50 mg/kg i.p. to fasted C57BL/6J male mice. The mice gradually developed higher levels of blood glucose from Day 1 to Day 4. The levels rose from 6.9 nmol/L to 13.1 nmol/L during the STZ induction period. Five days (Day 4) after the last STZ dosing, the mice were randomly distributed into 10 treatment groups each containing 10 mice in good condition. Treatment started on this day, before onset of diabetes and continued beyond the onset. The treatment groups were as shown in Table 5.

TABLE 5

| Group | Induction of | | Dose | |
|---|---|---|---|---|
| No. | Diabetes | Test Article | mg/kg | Administration |
| 1 | + | Vehicle | 0 | i.p. once daily |
| | | | | (QD) |
| 2 | + | KINERET ® | 100 | i.p. once daily |
| | | | | (QD) |
| 3 | + | KINERET ® | 30 | i.p. once daily |
| | | | | (QD) |
| 4 | + | I10-TripT-IL1-RA | 100 | i.p. once daily |
| | | | | (QD) |
| 5 | + | I10-TripT-IL1-RA | 30 | i.p. once daily |
| | | | | (QD) |
| 6 | + | I10-TripT-IL1-RA | 100 | i.p. twice weekly |
| | | | | (QD) |

The study period was 28 days and the mice were weighed once weekly during the treatment period. Blood glucose levels were measured every other day during the study period in order to monitor development of diabetes. A droplet of whole blood was collected by tail vein bleeding and placed on an Ascensia ELITE® blood glucose test strip and analyzed with an Ascensia ELITE® blood glucose meter (Bayer). The values were recorded, and x-fold increase in any given group compared to levels at treatment initiation was calculated. Clinical symptoms were observed daily or as appropriate in groups where adverse symptoms occurred.

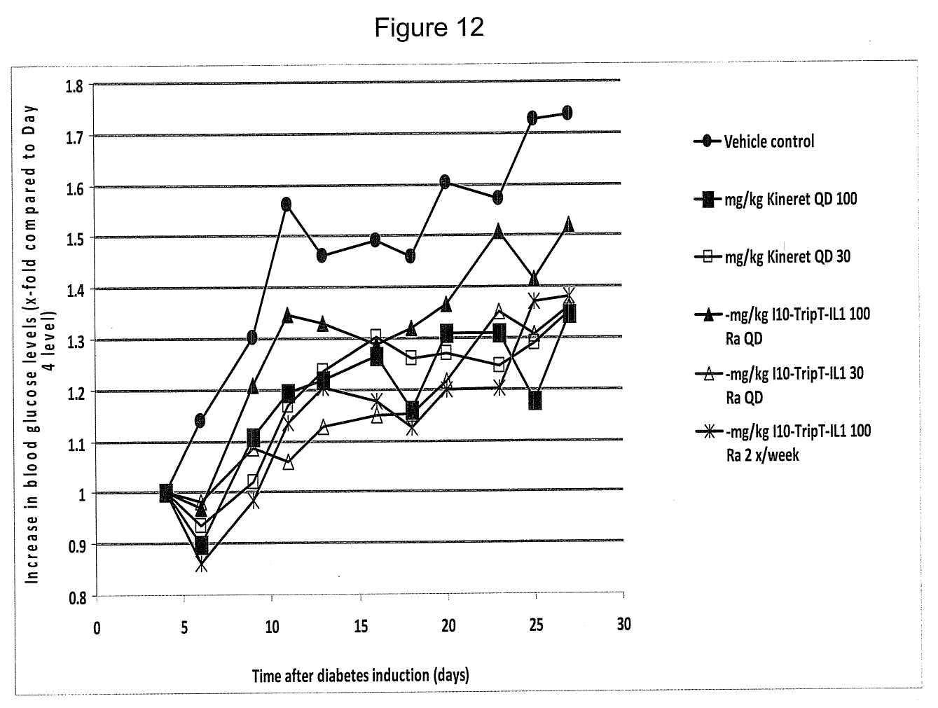

As shown in FIG. 12, a marked reduction of blood glucose levels was observed after daily i.p. dosing of either I10-TripT-IL1-Ra or KINERET® at both 100 and 30 mg/kg. Furthermore, twice weekly dosing of 100 mg/kg I10-TripT-IL1Ra was equally effective as daily dosing of 100 mg/kg KINERET®. These data demonstrate that trimerized IL-1Ra is an effective treatment of experimentally induced diabetes.

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in molecular biology, immunology, chemistry, biochemistry or in the relevant fields are intended to be within the scope of the appended claims.

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is to be noted that, as used herein and in the appended claims, the singular forms “a,” “an,” and “the” include the plural reference unless the context clearly dictates otherwise. Thus, for example, a reference to “a linker” is a reference to one or more linkers and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein.

Any numerical values recited herein include all values from the lower value to the upper value in increments of one unit provided that there is a separation of at least two units between any lower value and any higher value. As an example, if it is stated that the concentration of a component or value of a process variable such as, for example, size, angle size, pressure, time and the like, is, for example, from 1 to 90, specifically from 20 to 80, more specifically from 30 to 70, it is intended that values such as 15 to 85, 22 to 68, 43 to 51, 30 to 32, etc. are expressly enumerated in this specification. For values which are less than one, one unit is considered to be 0.0001, 0.001, 0.01 or 0.1 as appropriate. These are only examples of what is specifically intended and all possible combinations of numerical values between the lowest value and the highest value enumerated are to be considered to be expressly stated in this application in a similar manner.

Particular methods, devices, and materials are described, although any methods and materials similar or equivalent to those described herein can be used in the practice or testing of the invention. The disclosures of all references and publications cited above are expressly incorporated by reference in their entireties to the same extent as if each were incorporated by reference individually.

What is claimed is:

1. A fusion protein comprising a trimerizing domain and an IL-1Ra polypeptide that inhibits IL-1 activity.

2. The fusion protein of claim 1, wherein the IL-1Ra polypeptide is at least 85% identical to SEQ ID NO: 38 as the result of conservative amino acid substitution, and comprises Trp16, Gln20, Tyr34, Gln36 and Tyr147.

3. The fusion protein of claim 1, wherein the IL-1Ra polypeptide is at least 95% identical to SEQ ID NO: 38.

4. The fusion protein of claim 2 further comprising at least one mutation selected from the group consisting of D47N, E52R, E90Y, P38Y, H54R, Q129L and M136N.

5. The fusion protein of claim 1 wherein the trimerizing domain is derived from human tetranectin.

6. The fusion protein of claim 1, wherein the trimerizing domain is a tetranectin trimerizing structural element.

7. The fusion protein of claim 1, wherein the trimerizing domain is at least 66% identical to SEQ ID NO:1.

8. A trimeric complex comprising three fusion proteins of claim 5, wherein the fusion proteins are the same or different.

9. A trimeric complex comprising three fusion proteins of claim 6, wherein the fusion proteins are the same or different.

10. The fusion protein of claim 1, further comprising polyethylene glycol.

11. The fusion protein of claim 1, further comprising a linker between the IL-1Ra polypeptide and the trimerizing domain.

12. A trimeric complex comprising three fusion proteins, wherein each fusion protein comprises a fusion protein of claim 1, and wherein at least one of the fusion proteins is selected from the group consisting of TripK-IL-1ra (SEQ ID NO: 39); TripV-IL-1ra (SEQ ID NO: 40); TripT-IL-1ra (SEQ ID NO: 41); TripQ-IL-1ra (SEQ ID NO: 42); I10-TripK-IL-1ra (SEQ ID NO: 43); I10-TripV-IL-1ra (SEQ ID NO: 44); I10-TripT-IL-1ra (SEQ ID NO: 45); I10-TripQ-IL-1ra (SEQ ID NO: 46); V17-TripT-IL1Ra (SEQ ID NO: 55); V17-TripK-IL-1Ra (SEQ ID NO: 56); V17-TripV-IL-1RA (SEQ ID NO: 57); and V17-TripQ-IL1RA (SEQ ID NO: 58).

13. An isolated polynucleotide encoding the polypeptide of claim 1.

14. A vector comprising the polynucleotide of claim 13.

15. A host cell comprising the vector of claim 14.

16. A pharmaceutical composition comprising the fusion protein of claim 1 and at least one pharmaceutically acceptable excipient.

17. A pharmaceutical composition comprising the trimeric complex of claim 7 and least one pharmaceutically acceptable excipient.

18. A method for treating a disease mediated by interleukin 1 comprising administering to a patient in need thereof of the pharmaceutical composition of claim 17.

19. The method of claim 18, wherein the disease is an inflammatory disease.

20. The method of claim 19, wherein the inflammatory disease is rheumatoid arthritis.

21. The method of claim 19, wherein the inflammatory disease is diabetes.

22. The method of claim 19, further comprising administering to the patient, either simultaneously or sequentially, an anti-inflammatory agent.

23. The fusion protein of claim 1 further comprising an anti-inflammatory agent covalently linked to the fusion protein.

24. The method of claim 19 wherein at least one fusion protein is covalently linked to an anti-inflammatory agent.

25. A polypeptide complex comprising at least two fusion proteins of claim 1.

26. The polypeptide complex of claim 25 comprising at least four fusion proteins.


SEQUENCE LISTING (63 sequences)

SEQ ID NO: 1 - protein, 36 aa, Artifical sequence
  MISC_FEATURE (1)..(9): variable
XXXXXXXXXLXXXXXXLXXEVXXLKEXQALQTVCLX

SEQ ID NO: 2 - protein, 52 aa, Artifical sequence
  Synthetic
EPPTQKPKKIVNAKKDVVNTKMFEELKSRLDTLAQEVALLKEQQALQTVSLK

SEQ ID NO: 3 - protein, 51 aa, Artifical sequence
  Synthetic
EPPTQKPKKIVNAKKDVVNTKMFEELKSRLDTLAQEVALLKEQQALQTVSL

SEQ ID NO: 4 - protein, 50 aa, Artifical sequence
  Synthetic
EPPTQKPKKIVNAKKDVVNTKMFEELKSRLDTLAQEVALLKEQQALQTVS

SEQ ID NO: 5 - protein, 49 aa, Artifical sequence
  Synthetic
EPPTQKPKKIVNAKKDVVNTKMFEELKSRLDTLAQEVALLKEQQALQTV

SEQ ID NO: 6 - protein, 52 aa, Artifical sequence
  Synthetic
PPTQKPKKIVNAKKDVVNTKMFEELKSRLDTLAQEVALLKEQQALQTVSLKG

SEQ ID NO: 7 - protein, 51 aa, Artifical sequence
  Synthetic
PTQKPKKIVNAKKDVVNTKMFEELKSRLDTLAQEVALLKEQQALQTVSLKG

SEQ ID NO: 8 - protein, 50 aa, Artifical sequence
  Synthetic
TQKPKKIVNAKKDVVNTKMFEELKSRLDTLAQEVALLKEQQALQTVSLKG

SEQ ID NO: 9 - protein, 49 aa, Artifical sequence
  Synthetic
QKPKKIVNAKKDVVNTKMFEELKSRLDTLAQEVALLKEQQALQTVSLKG

SEQ ID NO: 10 - protein, 48 aa, Artifical sequence
  Synthetic
KPKKIVNAKKDVVNTKMFEELKSRLDTLAQEVALLKEQQALQTVSLKG

SEQ ID NO: 11 - protein, 47 aa, Artifical sequence
  Synthetic
PKKIVNAKKDVVNTKMFEELKSRLDTLAQEVALLKEQQALQTVSLKG

SEQ ID NO: 12 - protein, 46 aa, Artifical sequence
  Synthetic
KKIVNAKKDVVNTKMFEELKSRLDTLAQEVALLKEQQALQTVSLKG

SEQ ID NO: 13 - protein, 45 aa, Artifical sequence
  Synthetic
KIVNAKKDVVNTKMFEELKSRLDTLAQEVALLKEQQALQTVSLKG

SEQ ID NO: 14 - protein, 44 aa, Artifical sequence
  Synthetic
IVNAKKDVVNTKMFEELKSRLDTLAQEVALLKEQQALQTVSLKG

SEQ ID NO: 15 - protein, 43 aa, Artifical sequence
  Synthetic
VNAKKDVVNTKMFEELKSRLDTLAQEVALLKEQQALQTVSLKG

SEQ ID NO: 16 - protein, 42 aa, Artifical sequence
  Synthetic
NAKKDVVNTKMFEELKSRLDTLAQEVALLKEQQALQTVSLKG

SEQ ID NO: 17 - protein, 41 aa, Artifical sequence
  Synthetic
AKKDVVNTKMFEELKSRLDTLAQEVALLKEQQALQTVSLKG

SEQ ID NO: 18 - protein, 40 aa, Artifical sequence
  Synthetic
KKDVVNTKMFEELKSRLDTLAQEVALLKEQQALQTVSLKG

SEQ ID NO: 19 - protein, 39 aa, Artifical sequence
  Synthetic
KDVVNTKMFEELKSRLDTLAQEVALLKEQQALQTVSLKG

SEQ ID NO: 20 - protein, 37 aa, Artifical sequence
  Synthetic
VVNTKMFEELKSRLDTLAQEVALLKEQQALQTVSLKG

SEQ ID NO: 21 - protein, 37 aa, Artifical sequence; identical to SEQ ID NO: 20
  Synthetic

SEQ ID NO: 22 - protein, 36 aa, Artifical sequence
  Synthetic
VVNTKMFEELKSRLDTLAQEVALLKEQQALQTVSLK

SEQ ID NO: 23 - protein, 33 aa, Artifical sequence
  Synthetic
VVNTKMFEELKSRLDTLAQEVALLKEQQALQTV

SEQ ID NO: 24 - protein, 32 aa, Artifical sequence
  Synthetic
VVNTKMFEELKSRLDTLAQEVALLKEQQALQT

SEQ ID NO: 25 - protein, 30 aa, Artifical sequence
  Synthetic
VNTKMFEELKSRLDTLAQEVALLKEQQALQ

SEQ ID NO: 26 - protein, 35 aa, Artifical sequence
  Synthetic
NTKMFEELKSRLDTLAQEVALLKEQQALQTVSLKG

SEQ ID NO: 27 - protein, 34 aa, Artifical sequence
  Synthetic
TKMFEELKSRLDTLAQEVALLKEQQALQTVSLKG

SEQ ID NO: 28 - protein, 33 aa, Artifical sequence
  Synthetic
KMFEELKSRLDTLAQEVALLKEQQALQTVSLKG

SEQ ID NO: 29 - protein, 32 aa, Artifical sequence
  Synthetic
MFEELKSRLDTLAQEVALLKEQQALQTVSLKG

SEQ ID NO: 30 - protein, 52 aa, Artifical sequence
  Synthetic
EGPTQKPKKIVNAKKDVVNTKMFEELKSRLDTLAQEVALLKEQQALQTVSLK

SEQ ID NO: 31 - protein, 49 aa, Artifical sequence
  Synthetic
EGPTQKPKKIVNAKKDVVNTKMFEELKSRLDTLAQEVALLKEQQALQTV

SEQ ID NO: 32 - protein, 48 aa, Artifical sequence
  Synthetic
EGPTQKPKKIVNAKKDVVNTKMFEELKSRLDTLAQEVALLKEQQALQT

SEQ ID NO: 33 - protein, 47 aa, Artifical sequence
  Synthetic
EGPTQKPKKIVNAKKDVVNTKMFEELKSRLDTLAQEVALLKEQQALQ

SEQ ID NO: 34 - protein, 43 aa, Artifical sequence
  Synthetic
IVNAKKDVVNTKMFEELKSRLDTLAQEVALLKEQQALQTVSLK

SEQ ID NO: 35 - protein, 40 aa, Artifical sequence
  Synthetic
IVNAKKDVVNTKMFEELKSRLDTLAQEVALLKEQQALQTV

SEQ ID NO: 36 - protein, 39 aa, Artifical sequence
  Synthetic
IVNAKKDVVNTKMFEELKSRLDTLAQEVALLKEQQALQT

SEQ ID NO: 37 - protein, 38 aa, Artifical sequence
  Synthetic
IVNAKKDVVNTKMFEELKSRLDTLAQEVALLKEQQALQ

SEQ ID NO: 38 - protein, 152 aa, Homo sapiens
RPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 39 - protein, 204 aa, Artifical sequence
  Synthetic
EGPTQKPKKIVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 40 - protein, 200 aa, Artifical sequence [sequence omitted]

SEQ ID NO: 41 - protein, 200 aa, Artifical sequence
  Synthetic
EGPTQKPKKIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 42 - protein, 247 aa, Artifical sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 43 - protein, 195 aa, Artifical sequence
  Synthetic
IVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 44 - protein, 192 aa, Artifical sequence
  Synthetic
IVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 45 - protein, 191 aa, Artifical sequence
  Synthetic
IVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 46 - protein, 190 aa, Artifical sequence
  Synthetic
IVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 47 - protein, 252 aa, Artifical sequence [sequence omitted]

SEQ ID NO: 48 - protein, 249 aa, Artifical sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 49 - protein, 248 aa, Artifical sequence
  Synthetic
MVRANKRNEALRIESALLNKIAMLGTEKTAEGGSHHHHHHGSIEPDGGEGPTQKPKKIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 50 - protein, 247 aa, Artifical sequence; identical to SEQ ID NO: 42
  Synthetic

SEQ ID NO: 51 - protein, 241 aa, Artifical sequence
  Synthetic
MVRANKRNEALRIESALLNKIAMLGTEKTAEGGSHHHHHHGSIEPDIVNAKKDVVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 52 - protein, 238 aa, Artifical sequence
  Synthetic
MVRANKRNEALRIESALLNKIAMLGTEKTAEGGSHHHHHHGSIEPDIVNAKKDVVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 53 - protein, 237 aa, Artifical sequence
  Synthetic
MVRANKRNEALRIESALLNKIAMLGTEKTAEGGSHHHHHHGSIEPDIVNAKKDVVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 54 - protein, 236 aa, Artifical sequence
  Synthetic
MVRANKRNEALRIESALLNKIAMLGTEKTAEGGSHHHHHHGSIEPDIVNAKKDVVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 55 - protein, 188 aa, Artifical sequence
  Synthetic
VVNTKMFEELKSRLDTLAQEVALLKEQQALQTVSLKRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 56 - protein, 185 aa, Artifical sequence
  Synthetic
VVNTKMFEELKSRLDTLAQEVALLKEQQALQTVRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 57 - protein, 184 aa, Artifical sequence
  Synthetic
VVNTKMFEELKSRLDTLAQEVALLKEQQALQTRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 58 - protein, 183 aa, Artifical sequence
  Synthetic
VVNTKMFEELKSRLDTLAQEVALLKEQQALQRPSGRKSSKMQAFRIWDVNQKTFYLRNNQLVAGYLQGPNVNLEEKIDVVPIEPHALFLGIHGGKMCLSCVKSGDETRLQLEAVNITDLSENRKQDKRFAFIRSDSGPTTSFESAACPGWFLCTAMEADQPVSLTNMPDEGVMVTKFYFQEDE

SEQ ID NO: 59 - protein, 36 aa, Homo sapiens
VVNTKMFEELKSRLDTGAQEVALLKEQQALQTVCLK

SEQ ID NO: 60 - protein, 36 aa, Mus musculus
LVSSKMFEELKNRMDVLAQEVALLKEKQALQTVCLK

SEQ ID NO: 61 - protein, 36 aa, Bos taurus
RRVKEKDGDLKTQVEKLWREVNALKEMQALQTVCLR

SEQ ID NO: 62 - protein, 36 aa, Carcharodon carcharias
SKSGKGKDDLRNEIDKLWREVNSLKEMQALQTVCLK

SEQ ID NO: 63 - protein, 181 aa, Homo sapiens
EPPTQKPKKIVNAKKDVVNTKMFEELKSRLDTLAQEVALLKEQQALQTVCLKGTKVHMKCFLAFTQTKTFHEASEDCISRGGTLSTPQTGSEDDALTETLRQSVGNEAEIWLGLDDMAAEGTWVDMTGARIATKNWETEITAQPDGGKTEDCAVLSGAANGKWFDKRCRDQLPTICQFGIV